SEQUENCE LISTING (20 sequences)

SEQ ID NO: 1 - DNA, 15 bp, Artificial Sequence
  Synthetic oligonucleotide
tgctgctgctgctgc

SEQ ID NO: 2 - DNA, 11 bp, Artificial Sequence
  Synthetic oligonucleotide
tgctgctgctg

SEQ ID NO: 3 - DNA, 15 bp, Artificial Sequence
  Synthetic oligonucleotide
agcagcagcagcagc

SEQ ID NO: 4 - DNA, 11 bp, Artificial Sequence
  Synthetic oligonucleotide
agcagcagcag

SEQ ID NO: 5 - DNA, 14 bp, Artificial Sequence
  Synthetic oligonucleotide
aggcaggcaggcag

SEQ ID NO: 6 - DNA, 10 bp, Artificial Sequence
  Synthetic oligonucleotide
aggcaggcag

SEQ ID NO: 7 - DNA, 22 bp, Artificial Sequence
  Synthetic oligonucleotide
gctgctgctgctgctgctgctg

SEQ ID NO: 8 - DNA, 19 bp, Artificial Sequence
  Synthetic oligonucleotide
gctataccagcgtcgtcat

SEQ ID NO: 9 - DNA, 19 bp, Artificial Sequence
  Synthetic oligonucleotide
gctgctgctgctgctgctg

SEQ ID NO: 10 - DNA, 19 bp, Artificial Sequence
  Synthetic oligonucleotide
ctgctgctgctgctgctgc

SEQ ID NO: 11 - DNA, 19 bp, Artificial Sequence
  Synthetic oligonucleotide
tgctgctgctgctgctgct

SEQ ID NO: 12 - DNA, 16 bp, Artificial Sequence
  Synthetic oligonucleotide
gctgctgctgctgctg

SEQ ID NO: 13 - DNA, 13 bp, Artificial Sequence
  Synthetic oligonucleotide
gctgctgctgctg

SEQ ID NO: 14 - RNA, 19 nt, Artificial Sequence
  Synthetic oligonucleotide
gcugcugcugcugcugcug

SEQ ID NO: 15 - DNA, 20 bp, Artificial Sequence
  misc_feature (0)...(0): Primer
cgacagcgagtcagtgaatg

SEQ ID NO: 16 - DNA, 20 bp, Artificial Sequence
  Primer
accactctggcttcacaagg

SEQ ID NO: 17 - DNA, 19 bp, Artificial Sequence
  Synthetic oligonucleotide
tgctgctgctgctgctgct

SEQ ID NO: 18 - DNA, 19 bp, Artificial Sequence
  Synthetic oligonucleotide
gcagcagcagcagcagcag

SEQ ID NO: 19 - DNA, 19 bp, Artificial Sequence
  Synthetic oligonucleotide
agcagcagcagcagcagca

SEQ ID NO: 20 - DNA, 16 bp, Artificial Sequence
  Synthetic oligonucleotide
agcagcagcagcagca

(19) United States
(12) Patent Application Publication
(10) Pub. No.: US 2013/0059902 A1
(43) Pub. Date: Mar. 7, 2013

(54) METHODS AND COMPOSITIONS USEFUL IN TREATMENT OF DISEASES OR CONDITIONS RELATED TO REPEAT EXPANSION

(71) Applicants: David Corey, Dallas, TX (US); C. Frank Bennett, Carlsbad, CA (US); Eric E. Swayze, Encinitas, CA (US); Keith Gagnon, Grand Prairie, TX (US)

(72) Inventors: David Corey, Dallas, TX (US); C. Frank Bennett, Carlsbad, CA (US); Eric E. Swayze, Encinitas, CA (US); Keith Gagnon, Grand Prairie, TX (US)

(73) Assignee: Isis Pharmaceuticals, Inc., Carlsbad, CA (US)

(21) Appl. No.: 13/577,648

(22) PCT Filed: Feb. 8, 2011
(86) PCT No.: PCT/US11/24099

§ 371 Date: Nov. 20, 2012

Related U.S. Application Data
(60) Provisional application No. 61/405,157, filed on Oct. 20, 2010.
(60) Provisional application No. 61/302,454, filed on Feb. 8, 2010.
(60) Provisional application No. 61/302,450, filed on Feb. 8, 2010.

(51) Int. Cl. C07H 21/02; A61P 25/00; A61P 21/00; C12N 5/07; A61K 31/712

(52) U.S. Cl. 514/44 A; 536/24.5; 435/375

(57) ABSTRACT

The present invention is drawn to chemically-modified oligomers that are complementary to, and capable of hybridizing within the repeat region of CAG, CUG, or CCUG nucleotide repeat-containing RNAs (NRRs).

### GOVERNMENT SUPPORT

This invention was made with government support under 2-R01-GM073042-06 awarded by the National Institutes of Health. The government has certain rights in the invention.

### FIELD OF THE INVENTION

The present invention pertains generally to chemically-modified oligomers for use as therapeutics.

### SEQUENCE LISTING

The present application is being filed along with a Sequence Listing in electronic format.

The Sequence Listing is provided as a file entitled CORE0088WOSEQ.txt, created on Feb. 7, 2011 which is 8 Kb in size. The information in the electronic format of the sequence listing is incorporated herein by reference in its entirety.

### BACKGROUND OF THE INVENTION

Instability of gene-specific microsatellite and minisatellite repetitive sequences, leading to an increase in length of the repetitive sequences in the satellite, is associated with about 35 human genetic disorders. The causative gene for Huntington's disease, HD, is located on chromosome 4. Huntington's disease is inherited in an autosomal dominant fashion. When the gene has more than 35 CAG trinucleotide repeats coding for a polyglutamine stretch, the number of repeats can expand in successive generations. Because of the progressive increase in length of the repeats, the disease tends to increase in severity and presents at an earlier age in successive generations, a process called anticipation. The product of the HD gene is the 348 kDa cytoplasmic protein huntingtin. Huntingtin has a characteristic sequence of fewer than 40 glutamine amino acid residues in the normal form; the mutated huntingtin causing the disease has more than 40 residues. The continuous expression of mutant huntingtin molecules in neuronal cells results in the formation of large protein deposits which eventually give rise to cell death, especially in the frontal lobes and the basal ganglia (mainly in the caudate nucleus). The severity of the disease is generally proportional to the number of extra residues.

Unstable repeat units are also found in untranslated regions, such as in myotonic dystrophy type 1 (DM1) in the 3′ UTR or in intronic sequences such as in myotonic dystrophy type 2 (DM2). The normal number of repeats is around 5 to 37 for DMPK, but increases to premutation and full disease state two to ten fold or more, to 50, 100 and sometimes 1000 or more repeat units. For DM2/ZNF9 increases to 10,000 or more repeats have been reported. (Cleary and Pearson, Cytogenet. Genome Res. 100: 25-55, 2003).

DM1 is the most common muscular dystrophy in adults and is an inherited, progressive, degenerative, multisystemic disorder of predominantly skeletal muscle, heart and brain. DM1 is caused by expansion of an unstable trinucleotide (CTG)ₙ repeat in the 3′ untranslated region of the DMPK gene (myotonic dystrophy protein kinase) on human chromosome 19q (Brook et al, Cell, 1992). Type 2 myotonic dystrophy (DM2) is caused by a CCTG expansion in intron 1 of the ZNF9 gene, (Liquori et al, Science 2001). In the case of myotonic dystrophy type 1, the nuclearcytoplasmic export of DMPK transcripts is blocked by the increased length of the repeats, which form hairpin-like secondary structures that accumulate in nuclear foci. DMPK transcripts bearing a long (CUG)ₙ tract can form hairpin-like structures that bind proteins of the muscleblind family and subsequently aggregate in ribonuclear foci in the nucleus. These nuclear inclusions are thought to sequester muscleblind proteins, and potentially other factors, which then become limiting to the cell. In DM2, accumulation of ZNF9 RNA carrying the (CCUG)ₙ expanded repeat form similar foci. Since muscleblind proteins are splicing factors, their depletion results in a dramatic rearrangement in splicing of other transcripts, referred to as spliceopathy.

Transcripts of many genes consequently become aberrantly spliced, for instance by inclusion of fetal exons, or exclusion of exons, resulting in non-functional proteins and impaired cell function. For DM1, the aberrant transcript that accumulates in the nucleus could be down regulated or fully removed. Even relatively small reductions of the aberrant transcript could release substantial and possibly sufficient amounts of sequestered cellular factors and thereby help to restore normal RNA processing and cellular metabolism for DM (Kanadia et al., PNAS 2006).

### SUMMARY OF THE INVENTION

The present invention is drawn to chemically-modified oligomers that are complementary to, and capable of hybridizing within the repeat region of CAG, CUG, or CCUG nucleotide repeat-containing RNAs (NRRs). The chemically-modified oligonucleotides of the present invention are useful in the selective disruption of the deleterious effects of mutant NRRs to treat diseases, without affecting the normal function of the wild-type allele. The chemically-modified oligonucleotides described herein that target triplet CAG or CUG or quartet CCUG repeat sequences can incorporate one or more of the following criteria: (i) the oligonucleotide should have sufficient affinity toward the repeat expansion portion of the NRR, (ii) the oligonucleotide drug design motif (i.e., the pattern of nucleoside modifications incorporated into the antisense oligonucleotide) should minimizethe antisense oligonucleotides' propensity to form stable self complementary structures, or (iii) the oligonucleotides should be stable to both exo- and endonucleases. The chemically-modified oligonucleotides of the present invention are useful in the preferential lowering of, or the preferential inhibition of the function of, mutant versus wild-type forms of CAG, CUG, or CCUG repeat containing RNAs.

In an embodiment of the invention are provided chemically-modified oligonucleotides 17 to 22 nucleobases in length comprising a nucleobase sequence of SEQ ID NO: 1 [TGCTGCTGCTGCTGC] which is 100% complementary within a repeat region of a CAG nucleotide repeat containing RNA, wherein: each T is independently a uridine or thymidine nucleoside, or a nucleoside containing a uracil or thymine base or analogue thereof, and comprising an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base (e.g., a 5-methylcytosine), comprising a 2′-deoxyribose sugar; and one or both of the 5′ or 3′ terminal nucleosides comprises one or more nuclease-resistant modifications. In an embodiment, said one or more nuclease-resistant modifications is independently a modified sugar moiety or a modified internucleoside linkage. In an embodiment, the nuclease-resistant modification is independently a bicyclic sugar moiety. In certain chemically-modified oligonucleotides of the invention, the CAG nucleotide repeat containing RNA comprises 20 or more, 30 or more, or 40 or more repeats. In certain embodiments, the chemically-modified oligonucleotides provided herein are 17, 18, 19, 20, 21 or 22 nucleobases in length.

In an embodiment of the invention there are provided chemically-modified oligonucleotides 13 to 22 nucleobases in length comprising a nucleobase sequence of SEQ ID NO.: 2 [TGCTGCTGCTG] which is 100% complementary within a repeat region of a CAG nucleotide repeat containing RNA, wherein: each T is independently a uridine or thymidine nucleoside, or a nucleoside containing a uracil or thymine base or analogue thereof, and each comprising an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base (e.g., 5-methylcytosine), comprising a 2′-deoxyribose sugar; and one or both of the 5′ or 3′ terminal nucleosides comprises one or more nuclease-resistant modifications. In an embodiment, said one or more nuclease-resistant modifications is independently a modified sugar moiety or a modified internucleoside linkage.

In an embodiment, the nuclease-resistant modification is a bicyclic sugar moiety. In certain chemically-modified oligonucleotides of the invention, the CAG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In certain chemically-modified oligonucleotides of the invention, each of thymine (T) nucleobase can be independently replaced with a thymine analogue. In certain chemically-modified oligonucleotides of the invention, each of guanine (G) nucleobase can be independently replaced with a guanine analogue. In certain chemically-modified oligonucleotides of the invention, each of cytosine (C) nucleobase can be independently replaced with a cytosine analogue. In certain chemically-modified oligonucleotides of the invention, each of uracil (U) nucleobase can be independently replaced with a uracil analogue.

In one embodiment, each T is independently a thymidine or uridine nucleoside, or a nucleoside containing a uracil or thymine base or analogue thereof, comprising an independently selected bicyclic sugar moiety. In an additional embodiment, each T is independently a thymidine or uridine nucleoside, or a nucleoside containing a uracil or thymine base or analogue thereof, comprising an independently selected 4′ to 2′ bicyclic sugar moiety. In an embodiment, the 4′ to 2′ bridge comprises from 2 to 4 linked groups independently selected from —[C(Rₐ)(R_{b})]_{y}—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(R₁)—;

wherein:

x is 0, 1, or 2;

y is 1, 2, 3, or 4;

each Rₐ and R_{b} is, independently, H, a protecting group, hydroxyl, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₉ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₂₀ aryl, substituted C₅-C₉ aryl, acyl (C(═O)—H), substituted acyl, a heterocycle radical, a substituted heterocycle radical, C₁-C₆ aminoalkyl, substituted C₁-C₆ aminoalkyl or a protecting group.

In certain chemically-modified oligonucleotides of the invention, the bridge of the bicyclic sugar moiety is, —[C(R_{c})(R_{d})]ₙ—, —[C(R)(R_{d})]ₙ—O—, —C(R_{c}R_{d})—N(Rₑ)—O— or —C(R_{c}R_{d})—O—N(Rₑ)—, wherein each R_{c} and R_{d} is independently hydrogen, halogen, substituted or unsubstituted C₁-C₆ alkyl and each Rₑ is independently hydrogen or substituted or unsubstituted C₁-C₆ alkyl. In additional chemically-modified oligonucleotides of the invention each bicyclic sugar-modified thymidine nucleoside, the 4′ to 2′ bridge is independently a 4′-(CH₂)_{2-2′,4}′-(CH₂)_{3-2′,4}′-CH₂—O-2′,4′-CH(CH₃)—O-2′,4′-(CH₂)₂—O-2′,4′-CH₂—O—N(Rₑ)-2′ and 4′-CH₂—N(Rₑ)—O-2′-bridge. In certain chemically-modified oligonucleotides of the invention, each T comprises a 4′-CH(CH₃)—O-2′ bicyclic sugar moiety.

In certain chemically-modified oligonucleotide of the invention, each T is independently a thymidine or uridine nucleoside, or a nucleoside containing a uracil or thymine base or analogue thereof, comprising an independently selected 2′-modified sugar moiety. In certain embodiments, such 2′-modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties. In certain chemically-modified oligonucleotides of the invention, the 2′-modification is 2′-O-(2-methoxy)ethyl (2′-O—CH₂CH₂OCH₃).

In certain chemically-modified oligonucleotides of the invention, the nucleosides are linked by phosphate internucleoside linkages. In additional embodiments, the chemically-modified oligonucleotide comprises at least one phosphorothioate linkage. In certain embodiments, each internucleoside linkage is a phosphorothioate linkage.

In an embodiment of the invention there are provided chemically-modified oligonucleotides 17 to 22 nucleobases in length comprising a nucleobase sequence of SEQ ID NO.: 1 [TGCTGCTGCTGCTGC] which is 100% complementary within a repeat region of a CAG nucleotide repeat containing RNA, wherein: each non-terminal T is independently a uridine or thymidine nucleoside, or a nucleoside containing a uracil or thymine base or analogue thereof, comprising a 2′-deoxyribose sugar; each terminal T is independently a uridine or thymidine nucleoside, or a nucleoside containing a uracil or thymine base or analogue thereof, comprising a 2′-deoxyribose sugar and/or a nuclease resistant modification; each G is a guanosine nucleoside, or a nucleoside containing a guanine or guanine analogue base, comprising a high-affinity sugar modification; each non-terminal C is a cytidine nucleoside, or a nucleoside containing a cytosine or cytosine analogue base (e.g., 5-methylcytosine), comprising a 2′-deoxyribose sugar; and each terminal C is independently a cytidine nucleoside, or a nucleoside containing a cytosine or cytosine analogue base, including a 5-methylcytosine, comprising a 2′-deoxyribose sugar and/or one or more nuclease resistant modifications. In an additional embodiment, the chemically-modified oligonucleotides are further modified on one or both of the 5′ or 3′ end with one or more nuclease-resistant modifications, said nuclease-resistant modification is a modified sugar and/or a modified internucleoside linkage. In an embodiment, the nuclease-resistant modification is a bicyclic sugar moiety. In certain chemically-modified oligonucleotides of the invention, the CAG nucleotide repeat containing RNA comprises 20 or more, 30 or more, or 40 or more repeats. In certain embodiments, the chemically-modified oligonucleotides provided herein are 17, 18, 19, 20, 21 or 22 nucleobases in length.

In an embodiment of the invention are provided chemically-modified oligonucleotides 13 to 22 nucleobases in length comprising a nucleobase sequence of SEQ ID NO.: 2 [TGCTGCTGCTG] which is 100% complementary within a repeat region of a CAG nucleotide repeat containing RNA, wherein: each non-terminal T is independently a uridine or thymidine nucleoside, or a nucleoside containing a uracil or thymine base or analogue thereof, comprising a 2′-deoxyribose sugar; each terminal T is independently a uridine or thymidine nucleoside, or a nucleoside containing a uracil or thymine base or analogue thereof, comprising a 2′-deoxyribose sugar and/or a nuclease resistant modification; each G is a nucleoside, or a nucleoside containing a guanine or guanine analogue base, comprising a high-affinity sugar modification; each non-terminal C is a cytidine nucleoside, or a nucleoside containing a cytosine or cytosine analogue base (e.g., 5-methylcytosine, comprising a 2′-deoxyribose sugar; and each terminal C is independently a cytidine nucleoside, or a nucleoside containing a cytosine or cytosine analogue base (e.g., 5-methylcytosine), comprising a 2′-deoxyribose sugar and/or one or more nuclease resistant modifications. In an additional embodiment, the chemically-modified oligonucleotides independently comprise 5′ or 3′ terminal nucleosides having one or more nuclease-resistant modifications. In an embodiment, said nuclease-resistant modification is independently a modified sugar moiety and/or modified internucleoside linkage. In an embodiment, the nuclease-resistant modification is a bicyclic sugar moiety. In certain chemically-modified oligonucleotides of the invention, the CAG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In one embodiment, each G is a guanosine comprising an independently selected bicyclic sugar moiety. In an additional embodiment, each G is a guanosine comprising an independently selected 4′ to 2′ bicyclic sugar moiety. In certain embodiments, the 4′ to 2′ bridge comprises from 2 to 4 linked groups independently selected from —[C(Rₐ)(R_{b})]_{y}—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(R₁)—;

wherein:
x is 0, 1, or 2;
y is 1, 2, 3, or 4;
each Rₐ and R_{b} is, independently, H, a protecting group, hydroxyl, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₉ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₂₀ aryl, substituted C₅-C₉ aryl, acyl (C(═O)—H), substituted acyl, a heterocycle radical, a substituted heterocycle radical, C₁-C₆ aminoalkyl, substituted C₁-C₆ aminoalkyl or a protecting group.

In certain chemically-modified oligonucleotides of the invention, the bridge of the bicyclic sugar moiety is, —[C(R_{c})(R_{d})]ₙ—, —[C(R_{c})(R_{d})]ₙ—O—, —C(R_{c}R_{d})—N(Rₑ)—O— or —C(R_{c}R_{d})—O—N(Rₑ)—, wherein each R_{c} and R_{d} is independently hydrogen, halogen, substituted or unsubstituted C₁-C₆ alkyl and each Rₑ is independently hydrogen or substituted or unsubstituted C₁-C₆ alkyl. In additional chemically-modified oligonucleotides of the invention each bicyclic sugar-modified thymidine nucleoside, the 4′ to 2′ bridge is independently a 4′-(CH₂)₂-2′,4′-(CH₂)₃-2′,4′-CH₂—O-2′,4′-CH(CH₃)—O-2′,4′-(CH₂)₂—O-2′,4′-CH₂—O—N(Rₑ)-2′ and 4′-CH₂—N(Rₑ)—O-2′-bridge. In certain chemically-modified oligonucleotides of the invention, each T comprises a 4′-CH(CH₃)—O-2′ bicyclic sugar moiety.

In certain chemically-modified oligonucleotides of the invention, each G is a guanosine nucleoside, or a nucleoside containing a guanine or guanine analogue base, comprising an independently selected 2′-modified sugar moiety. In certain embodiments, such 2′-modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties. In certain chemically-modified oligonucleotides of the invention, the 2′-modification is 2′-O-(2-methoxy)ethyl (2′-O—CH₂CH₂OCH₃).

In an embodiment of the invention there are provided chemically-modified oligonucleotides 17 to 22 nucleobases in length comprising a nucleobase sequence of SEQ ID NO.: 3 [AGCAGCAGCAGCAGC] which is 100% complementary within a repeat region of a CUG nucleotide repeat containing RNA, wherein: each A is independently a adenosine nucleoside, or a nucleoside containing an adenine or adenine analogue base, comprises an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal G is a guanosine nucleoside, or a nucleoside containing a guanine or guanine analogue base, comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is independently a bicyclic sugar moiety. In certain chemically-modified oligonucleotides of the invention, the CUG nucleotide repeat containing RNA comprises 20 or more, 30 or more, or 40 or more repeats. In certain embodiments, the chemically-modified oligonucleotides provided herein are 17, 18, 19, 20, 21 or 22 nucleobases in length.

In an embodiment of the invention there are provided chemically-modified oligonucleotides 13 to 22 nucleobases in length comprising a nucleobase sequence of SEQ ID NO.: 4 [AGCAGCAGCAG] which is 100% complementary within a repeat region of a CUG nucleotide repeat containing RNA, wherein: each A is independently a adenosine nucleoside, or a nucleoside containing an adenine or adenine analogue base, comprises an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal G is a guanosine nucleoside, or a nucleoside containing a guanine or guanine analogue base, comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is a bicyclic sugar moiety. In certain chemically-modified oligonucleotides of the invention, the CUG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In an embodiment of the invention there are provided chemically-modified oligonucleotides 17 to 22 nucleobases in length comprising a nucleobase sequence of SEQ ID NO.: 5 [AGGCAGGCAGGCAG] which is 100% complementary within a repeat region of a CCUG nucleotide repeat containing RNA, wherein: each A is independently a adenosine nucleoside, or a nucleoside containing an adenine or adenine analogue base, comprises an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal G is a guanosine nucleoside, or a nucleoside containing a guanine or guanine analogue base, comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is independently a bicyclic sugar moiety. In certain chemically-modified oligonucleotides of the invention, the CUG nucleotide repeat containing RNA comprises 20 or more, 30 or more, or 40 or more repeats. In certain embodiments, the chemically-modified oligonucleotides provided herein are 17, 18, 19, 20, 21 or 22 nucleobases in length.

In an embodiment of the invention are provided chemically-modified oligonucleotides 13 to 22 nucleobases in length comprising a nucleobase sequence of SEQ ID NO.: 6 [AGGCAGGCAG] which is 100% complementary within a repeat region of a CCUG nucleotide repeat containing RNA, wherein: each A is independently a adenosine nucleoside, or a nucleoside containing an adenine or adenine analogue base, comprises an independently selected high-affinity sugar modification; each non-terminal G is a guanosine nucleoside, or a nucleoside containing a guanine or guanine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal G is a guanosine nucleoside, or a nucleoside containing a guanine or guanine analogue base, comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is a bicyclic sugar moiety. In certain chemically-modified oligonucleotides of the invention, the CCUG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In certain chemically-modified oligonucleotides of the invention, each A nucleobase can be independently replaced with a adenine analogue. In certain chemically-modified oligonucleotides of the invention, each guanine nucleobase can be independently replaced with a guanine analogue. In certain chemically-modified oligonucleotides of the invention, each cytosine nucleobase can be independently replaced with a cytosine analogue.

In one embodiment, each A is an adenosine nucleoside, or a nucleoside containing an adenine or adenine analogue base, independently comprising a bicyclic sugar moiety. In an additional embodiment, each A is an adenosine nucleoside, or a nucleoside containing an adenine or adenine analogue base, independently comprising a 4′ to 2′ bicyclic sugar moiety. In an embodiment, the 4′ to 2′ bridge comprises from 2 to 4 linked groups independently selected from —[C(Ra)(Rb)]y-, C(Ra)═C(Rb)—, C(Ra)═N—, C(═NRa)-, —C(═O)—, —C(═S)—, —O—, —Si(Ra)2-, —S(═O)x-, and N(R1)-;
wherein:

x is 0, 1, or 2;

y is 1, 2, 3, or 4;

each Ra and Rb is, independently, H, a protecting group, hydroxyl, C1-C6 alkyl, substituted C1-C6 alkyl, C2-C6 alkenyl, substituted C2-C6 alkenyl, C2-C6 alkynyl, substituted C2-C6 alkynyl, C5-C9 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and

each J1 and J2 is, independently, H, C1-C6 alkyl, substituted C1-C6 alkyl, C2-C6 alkenyl, substituted C2-C6 alkenyl, C2-C6 alkynyl, substituted C2-C6 alkynyl, C5-C20 aryl, substituted C5-C9 aryl, acyl (C(═O)—H), substituted acyl, a heterocycle radical, a substituted heterocycle radical, C1-C6 aminoalkyl, substituted C1-C6 aminoalkyl or a protecting group.

In certain chemically-modified oligonucleotides of the invention, the bridge of the bicyclic sugar moiety is, [C(Rc)(Rd)]n-, [C(Rc)(Rd)]n-O—, C(RcRd)—N(Re)—O— or —C(RcRd)-O—N(Re)—, wherein each Rc and Rd is independently hydrogen, halogen, substituted or unsubstituted C1-C6 alkyl and each Re is independently hydrogen or substituted or unsubstituted C1-C6 alkyl. In additional chemically-modified oligonucleotides of the invention each bicyclic sugar-modified thymidine nucleoside, the 4′ to 2′ bridge is independently a 4′-(CH2)2-2′,4′-(CH2)3-2′,4′-CH2-O-2′,4′-CH(CH3)-O-2′,4′-(CH2)2-O-2′,4′-CH2-O—N(Rₑ)-2′ and 4′-CH2-N(Rₑ)—O-2′-bridge. In certain chemically-modified oligonucleotides of the invention, each A comprises a 4′-CH(CH3)-O-2′ bicyclic sugar moiety.

In certain chemically-modified oligonucleotide of the invention, each A is an adenosine nucleoside, or a nucleoside containing an adenine or adenine analogue base, independently comprising a 2′-modified sugar moiety. In certain embodiments, such 2′-modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH2)nO]mCH3, O(CH2)nNH2, O(CH2)nCH3, O(CH2)nONH2, OCH2C(═O)N(H)CH3, and O(CH2)nON[(CH2)nCH3]2, where n and m are from 1 to about 10. Other 2′-substituent groups can also be selected from: C1-C12 alkyl, substituted alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, poly-alkylamino, substituted silyl, an RNA cleaving group, a reporter group, an intercalator, a group for improving pharmacokinetic properties, or a group for improving the pharmacodynamic properties of an oligomeric compound, and other substituents having similar properties. In certain chemically-modified oligonucleotides of the invention, the 2′-modification is 2′-O-(2-methoxy)ethyl (2′-O—CH2CH2OCH3).

In certain chemically-modified oligonucleotides of the invention, the nucleosides are linked by phosphate internucleoside linkages. In additional embodiments, the chemically-modified oligonucleotide comprises at least one phosphorothioate linkage. In certain embodiments, each internucleoside linkage is a phosphorothioate linkage.

In an embodiment of the invention there are provided chemically-modified oligonucleotides 17 to 22 nucleobases in length comprising a nucleobase sequence of SEQ ID NO.: 3 [AGCAGCAGCAGCAGC] which is 100% complementary within a repeat region of a CUG nucleotide repeat containing RNA, wherein: each A is independently an adenosine nucleoside, or a nucleoside containing an adenine or adenine analogue base, comprising a 2′-deoxyribose sugar; each G is independently a guanosine nucleoside, or a nucleoside containing a guanine or guanine analogue base, comprising a high-affinity sugar modification; and each C is a cytidine nucleoside, or a nucleoside containing a cytosine or cytosine analogue base, including a 5-methylcytosine, comprises a 2′-deoxyribose sugar. In an additional embodiment, the chemically-modified oligonucleotides are further modified on one or both of the 5′ or 3′ end with one or more nuclease-resistant nucleotide, said nuclease-resistant nucleotide comprises a modified sugar and/or internucleoside linkage. In an embodiment, the nuclease-resistant nucleotides comprise a bicyclic sugar moiety. In certain chemically-modified oligonucleotides of the invention, the CUG nucleotide repeat containing RNA comprises 20 or more, 30 or more, or or more repeats. In certain embodiments, the chemically-modified oligonucleotides provided herein are 17, 18, 19, 20, 21 or 22 nucleobases in length.

In an embodiment of the invention there are provided chemically-modified oligonucleotides 13 to 22 nucleobases in length comprising a nucleobase sequence of SEQ ID NO.: 4 [GCAGCAGCAG] which is 100% complementary within a repeat region of a CUG nucleotide repeat containing RNA, wherein: each non-terminal A is an adenosine nucleoside, or a nucleoside containing an adenine or adenine analogue base, comprising a 2′-deoxyribose sugar; each terminal A is an adenosine nucleoside, or a nucleoside containing an adenine or adenine analogue base, comprising a 2′-deoxyribose sugar and/or a nuclease resistant modification; each G is a guanosine nucleoside, or a nucleoside containing a guanine or guanine analogue base, comprising an independently selected high-affinity sugar modification; each non-terminal C is a cytidine nucleoside, or a nucleoside containing a cytosine or cytosine analogue base, including a 5-methylcytosine, comprising a 2′-deoxyribose sugar; and each terminal C is a cytidine nucleoside, or a nucleoside containing a cytosine or cytosine analogue base (e.g., 5-methylcytosine), comprising independently a 2′-deoxyribose sugar and/or a nuclease resistant modification. In an embodiment, said nuclease-resistant modification is independently a modified sugar moiety and/or modified internucleoside linkage. In an embodiment, the nuclease-resistant modification is a bicyclic sugar moiety. In certain chemically-modified oligonucleotides of the invention, the CUG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In an embodiment of the invention there are provided chemically-modified oligonucleotides 17 to 22 nucleobases in length comprising a nucleobase sequence of SEQ ID NO.: 5 [AGGCAGGCAGGCAG] which is 100% complementary within a repeat region of a CCUG nucleotide repeat containing RNA, wherein: each G is independently a guanosine nucleoside, or a nucleoside containing a guanine or guanine analogue base, comprises an independently selected high-affinity sugar modification; each non-terminal A is an adenosine nucleoside, or a nucleoside containing an adenine or adenine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal A is a adenosine nucleoside, or a nucleoside containing an adenine or adenine analogue base, comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is independently a bicyclic sugar moiety. In certain chemically-modified oligonucleotides of the invention, the CUG nucleotide repeat containing RNA comprises 20 or more, 30 or more, or 40 or more repeats. In certain embodiments, the chemically-modified oligonucleotides provided herein are 17, 18, 19, 20, 21 or 22 nucleobases in length.

In an embodiment of the invention there are provided chemically-modified oligonucleotides 13 to 22 nucleobases in length comprising a nucleobase sequence of SEQ ID NO.: 6 [AGGCAGGCAG] which is 100% complementary within a repeat region of a CCUG nucleotide repeat containing RNA, wherein: each G is independently a guanosine nucleoside, or a nucleoside containing a guanine or guanine analogue base, comprises an independently selected high-affinity sugar modification; each non-terminal A is an adenoside nucleoside, or a nucleoside containing an adenine or adenine analogue base, comprising a 2′-deoxyribose sugar; each non-terminal C is a cytidine nucleoside, or a nucleoside containing a cytosine or cytosine analogue base, including a 5-methylcytosine, comprising a 2′-deoxyribose sugar; each terminal A is adenosine nucleoside comprising an independently selected 2′-deoxyribose sugar and/or nuclease resistant modification; and each terminal C is a cytidine nucleoside, or a nucleoside containing a cytosine or cytosine analogue base (e.g., 5-methylcytosine), comprising an independently selected 2′-deoxyribose sugar and/or nuclease resistant modification. In an embodiment, said one or more nuclease-resistant modifications is independently a modified sugar moiety and/or a modified internucleoside linkage. In an embodiment, the nuclease-resistant modification is a bicyclic sugar moiety. In certain chemically-modified oligonucleotides of the invention, the CCUG nucleotide repeat containing RNA comprises 20 or more, 30 or more, or 40 or more repeats. In certain embodiments, the chemically-modified oligonucleotides provided herein are 13, 14, 15, 16, 17, 18, 19, 20, 21 or 22 nucleobases in length.

In one embodiment, each G is independently a bicyclic sugar guanine nucleoside, or a nucleoside containing a guanine or guanine analogue base. In an additional embodiment, each G is independently a 4′ to 2′ bicyclic sugar nucleoside. In 4′ to 2′ bridge comprises from 2 to 4 linked groups independently selected from —[C(Ra)(Rb)]y—, C(Ra)═C(Rb)—, C(Ra)═N—, C(═NRa)-, —C(═O)—, —C(═S)—, —O—, —Si(Ra)2-, —S(═O)x—, and N(R1)-;

wherein:

x is 0, 1, or 2;

y is 1, 2, 3, or 4;

each Ra and Rb is, independently, H, a protecting group, hydroxyl, C1-C6 alkyl, substituted C1-C6 alkyl, C2-C6 alkenyl, substituted C2-C6 alkenyl, C2-C6 alkynyl, substituted C2-C6 alkynyl, C5-C9 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and

each J1 and J2 is, independently, H, C1-C6 alkyl, substituted C1-C6 alkyl, C2-C6 alkenyl, substituted C2-C6 alkenyl, C2-C6 alkynyl, substituted C2-C6 alkynyl, C5-C20 aryl, substituted C5-C9 aryl, acyl (C(═O)—H), substituted acyl, a heterocycle radical, a substituted heterocycle radical, C1-C6 aminoalkyl, substituted C1-C6 aminoalkyl or a protecting group.

In certain chemically-modified oligonucleotides of the invention, the bridge of the bicyclic sugar moiety is, [C(Rc)(Rd)]n—, [C(Rc)(Rd)]n—O—, C(RcRd)—N(Re)—O— or —C(RcRd)—O—N(Re)—, wherein each Rc and Rd is independently hydrogen, halogen, substituted or unsubstituted C1-C6 alkyl and each Re is independently hydrogen or substituted or unsubstituted C1-C6 alkyl. In additional chemically-modified oligonucleotides of the invention each bicyclic sugar-modified thymidine nucleoside, the 4′ to 2′ bridge is independently a 4′-(CH2)2-2′,4′-(CH2)3-2′,4′-CH2-O-2′,4′-CH(CH3)-O-2′,4′-(CH2)2-O-2′,4′-CH2-O—N(Re)-2′ and 4′-CH2-N(Re)—O-2′-bridge. In certain chemically-modified oligonucleotides of the invention, each G comprises a 4′-CH(CH3)-O-2′ bicyclic sugar moiety.

In certain chemically-modified oligonucleotides of the invention, each G is a guanosine nucleoside, or a nucleoside containing a guanine or guanine analogue base, comprising an independently selected 2′-modified sugar moiety. In certain embodiments, such 2′-modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH2)nO]mCH3, O(CH2)nNH2, O(CH2)nCH3, O(CH2)nONH2, OCH2C(═O)N(H)CH3, and O(CH2)nON[(CH2)nCH3]2, where n and m are from 1 to about 10. Other 2′-substituent groups can also be selected from: C1-C12 alkyl, substituted alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, poly-alkylamino, substituted silyl, an RNA cleaving group, a reporter group, an intercalator, a group for improving pharmacokinetic properties, or a group for improving the pharmacodynamic properties of an oligomeric compound, and other substituents having similar properties. In certain chemically-modified oligonucleotides of the invention, the 2′-modification is 2′-O-(2-methoxy)ethyl (2′-O—CH2CH2OCH3).

In certain chemically-modified oligonucleotides of the invention, the nucleosides are linked by phosphate internucleoside linkages. In additional embodiments, the chemically-modified oligonucleotide comprises at least one phosphorothioate linkage. In certain embodiments, each internucleoside linkage is a phosphorothioate linkage.

In an additional embodiment of the invention there are provided methods of selectively inhibiting the function of a mutant nucleotide repeat containing RNA in a cell, comprising contacting a cell having or suspected having a mutant nucleotide repeat containing RNA with any chemically-modified oligonucleotide described herein. In certain embodiments of the invention are methods of treating patient diagnosed with a disease or disorder associated with an RNA molecule containing a CAG triplet repeat expansion, comprising: administering to a patient diagnosed with said disease or disorder any chemically-modified oligonucleotide described herein. In an embodiment the disease or disorder is selected from Huntington's Disease, Atrophin 1 (DRPLA), Spinobulbar muscular atrophy/Kennedy disease, Spinocerebellar ataxia (SCA)1, SCA2, SCA3, SCA6, SCA7, SCA12, SCA17 or Huntington Disease-Like 2 (HDL2). In certain embodiments of the invention are methods of treating patient diagnosed with a disease or disorder associated with an RNA molecule containing a CUG triplet repeat expansion, comprising: administering to a patient diagnosed with said disease or disorder with any chemically-modified oligonucleotide described herein targeting a CUG repeat containing RNA. In an embodiment the disease or disorder is selected from Ataxin 8 opposite strand (ATXN80S), Huntinton disease-like 2, myotonic dystrophy, or SCA8. In certain embodiments of the invention are methods of treating patient diagnosed with a disease or disorder associated with an RNA molecule containing a CCUG repeat expansion, comprising: administering to a patient diagnosed with said disease or disorder with any chemically-modified oligonucleotide described herein targeting a CCUG repeat containing RNA. In an embodiment the disease or disorder is DM2.

In certain embodiments, the administering is done by intravenous injection, subcutaneous injection, intramuscular injection, intrathecal injection, or intracerebral injection. In certain embodiments, the administering is performed by intramuscular injection.

### DETAILED DESCRIPTION

As used herein, the term “nucleotide repeat-containing RNA” (NRR) means a mutant RNA molecule that contains a sequence of nucleotides comprising a repeat element wherein a triplet or quartet of nucleotides is repeated consecutively several times within said sequence affecting the normal processing of said RNA. These NRRs are also referred to in the art as “gain-of-function RNAs” that gain the ability to sequester hnRNPs and impair the normal action of RNA processing in the nucleus (see Cooper, T. (2009) Cell 136, 777-793; O'Rourke, J R (2009) J. Biol. Chem. 284 (12), 7419-7423), which are herein incorporated by reference in the entirety. Several disease states are associated with NRRs, some of said diseases only occurring where a threshold number of repeats are contained within the NRR. For instance, one disease state might be caused by 50-200 repeats in a particular gene, where a different disease or severity is caused by a different number of repeats >400 in the same gene. Some mutations that cause NRRs can be heterozygous and therefore some copies of the gene can be functional and as a result, there is a need to interfere with the mutant NRR without affecting the wild type copy of the gene. Examples of CAG, CUG, and CCUG nucleotide repeat-containing RNA molecules implicated in disease are the following:

| | | | COPY | COPY | |
|---|---|---|---|---|---|
| | | AFFECTED | NUMBER | NUMBER | |
| DISEASE | REPEAT | GENE | (NORMAL) | (DISEASED) | Reference |
| Atrophin 1 | CAG | ATN1/DRPLA | 7 to 34 | 49-93 | Nat. Genet. 10: 99, |
| (DRPLA) | | | | | 1995 |
| Huntington | CAG | Htt | <28 | >36 | Lancet 369: 220, |
| disease | | | | | 2007 |
| Huntington | CAG | junctophilin-3 | 6 to 28 | 44 to 57 | Nat. Clin Prac Neurol. |
| disease-like 2 | | (JPH3) | | | 3: 517, 2007 |
| (HDL2) | | | | | |
| Spinal and bulbar | CAG | Androgen | 10 to 36 | 38 to 62 | Nature 352: 77, 1991 |
| muscular | | receptor (AR) | | | |
| atrophy/Kennedy | | (X-linked) | | | |
| disease | | | | | |
| Spinocerebellar | CAG | ataxin-1 | 6 to 35 | 49 to 88 | NCBI/OMIM |
| ataxia 1 | | (ATXN1) | | | |
| Spinocerebellar | CAG | protein | 9 to 28 | 55 to 78 | Brain Res Bull. 56: |
| ataxia 12 | | phosphatase | | | 397, 2001 |
| | | PP2A | | | |
| | | (PPP2R2B) | | | |
| | | | 7 to 28 | 66 to 78 | Wikipedia |
| Spinocerebellar | CAG | TATA box- | 25 to 42 | 47 to 63 | Eur. J. Hum. Genet. |
| ataxia | | binding protein | | | 9: 160, 2001 |
| 17/Huntington | | (TBP) | | | (NCBI/OMIM) |
| disease-like 4 | | | | | |
| (HDL4) | | | | | |
| Spinocerebellar | CAG | ATXN2 | 17 to 29 | 37 to 50 | Nat. Genet. 14: 285, |
| ataxia 2 | | | | | 1996 (NCBI/OMIM) |
| Spinocerebellar | CAG | ATXN3 | 15 to 34 | 35 to 59 | Nat. Genet. 14: 277, |
| ataxia 3 | | | | | 1996(NCBI/OMIM) |
| (Machado-Joseph | | | 14 to 32 | 33 to 77 | Wikipedia |
| disease | | | 10 to 51 | 55-87 | Human Mol. Genet. |
| | | | | | 17: 2071, 2008 |
| | | | | | (NCBI/OMIM) |
| | | | 12 to 40 | 55 to 86 | Wikipedia |
| Spinocerebellar | CAG | CACNA1A | 4 to 18 | 21 to 30 | Wikipedia |
| ataxia 6 | | | | | |
| | | | 5 to 20 | 21 to 25 | Am. J. Hum. Genet. |
| | | | | | 61: 336, 1997 |
| | | | | | (NCBI/OMIM) |
| Spinocerebellar | CAG | ATXN7 | 7 to 17 | 38-130 | Nat. Genet. 17: 65, |
| ataxia 7/OPCA3 | | | | | 1997 (NCBI/OMIM) |
| Ataxin 8 opposite | CUG with or | SCA8/ataxin 8 | 16-37 | 107-127 | Nat. Genet 21: 379, |
| strand | without | | | | 1999 (NCBI/OMIM) |
| (ATXN8OS) | interruptions | | | | |
| Huntington | CAG/CUG | junctophilin-3 | 6 to 28 | 44 to 57 | Nat. Clin Prac Neurol. |
| disease-like 2 | | (JPH3) | | | 3: 517, 2007 |
| (HDL2) | | | | | |
| Myotonic | CUG | DMPK | 5 TO 35 | 80 TO >2500 | Harper, Myotonic |
| dystrophy (DM1) | | | | | Dystrophy (Saunders, |
| | | | | | London, ed.3, 2001) |
| | | | | 50 to >3500 | Annu. Rev. Neurosci. |
| | | | | | 29: 259, 2006 |
| | | | 5 to 37 | >50 | EMBO J. 19: 4439, |
| | | | | | 2000 |
| | | | | 50 to >2000 | Curr Opin Neurol. 20: |
| | | | | | 572, 2007 |
| DM2 | CCUG | zinc finger | | 75 to 11,000 | Science 293: 864, |
| | | protein-9 | | | 2001 (NCBI/OMIM) |
| Spinocerebellar | CUG | SCA8 | | 74 to >1300 | Nat. Genet. 21: 379, |
| ataxia 8 | | | | | 1999 |

### DEFINITIONS

Unless otherwise indicated, the following terms have the following meanings:

As used herein, “nucleoside” refers to a compound comprising a heterocyclic base moiety and a sugar moiety. Nucleosides include, but are not limited to, naturally occurring nucleosides (as found in DNA and RNA), abasic nucleosides, modified nucleosides, and sugar-modified nucleosides. Nucleosides may be modified with any of a variety of substituents.

As used herein, “sugar moiety” means a natural (furanosyl), a modified sugar moiety or a sugar surrogate.

As used herein, “modified sugar moiety” means a chemically-modified furanosyl sugar or a non-furanosyl sugar moiety. Also, embraced by this term are furanosyl sugar analogs and derivatives including bicyclic sugars, tetrahydropyrans, morpholinos, 2′-modified sugars, 4′-modified sugars, 5′-modified sugars, and 4′-substituted sugars.

As used herein, “sugar-modified nucleoside” means a nucleoside comprising a modified sugar moiety.

As used herein the term “sugar surrogate” refers to a structure that is capable of replacing the furanose ring of a naturally occurring nucleoside. In certain embodiments, sugar surrogates are non-furanose (or 4′-substituted furanose) rings or ring systems or open systems. Such structures include simple changes relative to the natural furanose ring, such as a six membered ring or may be more complicated as is the case with the non-ring system used in peptide nucleic acid. Sugar surrogates includes without limitation morpholinos and cyclohexenyls and cyclohexitols. In most nucleosides having a sugar surrogate group the heterocyclic base moiety is generally maintained to permit hybridization.

As used herein, “nucleotide” refers to a nucleoside further comprising a modified or unmodified phosphate linking group or a non-phosphate internucleoside linkage.

As used herein, “linked nucleosides” may or may not be linked by phosphate linkages and thus includes “linked nucleotides.”

As used herein, “nucleobase” refers to the heterocyclic base portion of a nucleoside. Nucleobases may be naturally occurring or may be modified and therefore include, but are not limited to adenine, cytosine, guanine, uracil, thymidine and analogues thereof such as 5-methylcytosine. In certain embodiments, a nucleobase may comprise any atom or group of atoms capable of hydrogen bonding to a base of another nucleic acid.

As used herein, “modified nucleoside” refers to a nucleoside comprising at least one modification compared to naturally occurring RNA or DNA nucleosides. Such modification may be at the sugar moiety and/or at the nucleobases.

As used herein, “Tₘ” means melting temperature which is the temperature at which the two strands of a duplex nucleic acid separate. Tₘ is often used as a measure of duplex stability or the binding affinity of an antisense compound toward a complementary RNA molecule.

As used herein, a “high-affinity sugar modification” is a modified sugar moiety which when it is included in a nucleoside and said nucleoside is incorporated into an antisense oligonucleotide, the stability (as measured by Tₘ) of said antisense oligonucleotide: RNA duplex is increased as compared to the stability of a DNA:RNA duplex.

As used herein, a “high-affinity sugar-modified nucleoside” is a nucleoside comprising a modified sugar moiety that when said nucleoside is incorporated into an antisense compound, the binding affinity (as measured by Tₘ) of said antisense compound toward a complementary RNA molecule is increased. In certain embodiments of the invention at least one of said sugar-modified high-affinity nucleosides confers a ΔTₘ of at least 1 to 4 degrees per nucleoside against a complementary RNA as determined in accordance with the methodology described in Freier et al., Nucleic Acids Res., 1997, 25, 4429-4443, which is incorporated by reference in its entirety. In another aspect, at least one of the high-affinity sugar modifications confers about 2 or more, 3 or more, or 4 or more degrees per modification. In the context of the present invention, examples of sugar-modified high affinity nucleosides include, but are not limited to, (i) certain 2′-modified nucleosides, including 2′-substituted and 4′ to 2′ bicyclic nucleosides, and (ii) certain other non-ribofuranosyl nucleosides which provide a per modification increase in binding affinity such as modified tetrahydropyran and tricycloDNA nucleosides. For other modifications that are sugar-modified high-affinity nucleosides see Freier et al., Nucleic Acids Res., 1997, 25, 4429-4443.

As used herein, a “nuclease resistant modification” means a sugar modification or modified internucleoside linkage which, when incorporated into an oligonucleotide, makes said oligonucleotide more stable to degradation under cellular nucleases (e.g. exo- or endo-nucleases). Examples of nuclease resistant modifications include, but are not limited to, phosphorothioate internucleoside linkages, bicyclic sugar modifications, 2′-modified nucleotides, or neutral internucleoside linkages.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, oligomeric compounds provided herein include one or more bicyclic nucleosides wherein the bridge comprises a 4′ to 2′ bicyclic nucleoside. Examples of such 4′ to 2′ bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya, et al., J. Org. Chem., 2009, 74, 118-134); and 4′-CH₂—C—(═CH₂)-2′ (and analogs thereof see published International Application WO 2008/154401, published on Dec. 8, 2008). See, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. Nos. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′,4′-(CH₂)_{2-2′,4}′-(CH₂)_{3-2′,4}′-CH₂—O-2′,4′-(CH₂)₂—O-2′,4′-CH₂—O—N(R)-2′ and 4′-CH₂—N(R)—O-2′— wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃₋₂′) BNA, and (K) ethylene carbocyclic (4′-CH₂—CH₂-2′) (carba LNA or “cLNA”) as depicted below.

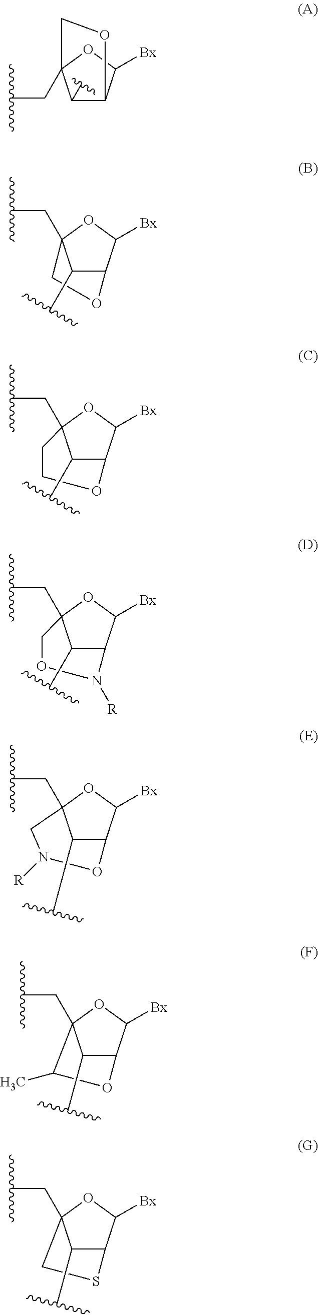

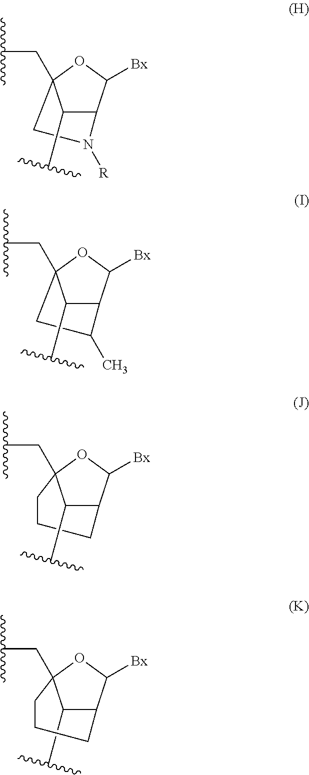

wherein Bx is the base moiety and R is independently H, a protecting group or C₁-C₁₂ alkyl.

In certain embodiments, bicyclic nucleoside include Formula I:

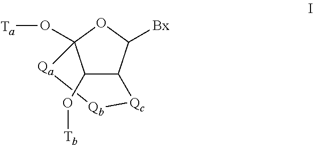

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R)—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleoside include Formula II:

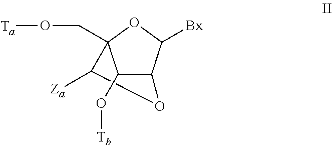

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleoside include Formula III:

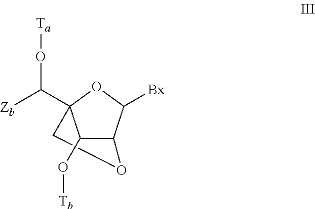

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleoside include Formula IV:

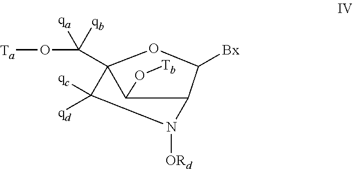

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleoside include Formula V:

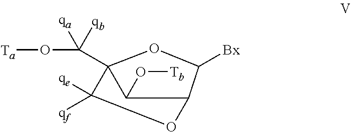

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methylcytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-Amino- and 2′-methylamino-BNA's have been prepared and the thermal stability of their duplexes with complementary RNA and DNA strands has been previously reported.

In certain embodiments, bicyclic nucleoside include Formula VI:

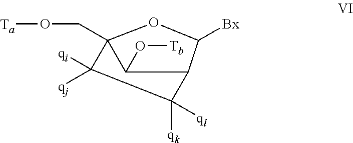

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃₋₂′ bridge and the alkenyl analog bridge 4′-CH═CH—CH₂-2′ has been described (Frier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides. Modified THP nucleosides include, but are not limited to, what is referred to in the art as hexitol nucleic acid (HNA), anitol nucleic acid (ANA), manitol nucleic acid (MNA) (see Leumann, C J. Bioorg. & Med. Chem. (2002) 10:841-854), fluoro HNA (F-HNA) or those compounds having Formula X:

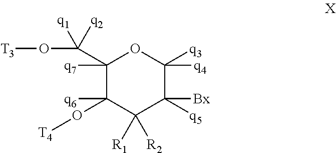

wherein independently for each of said at least one tetrahydropyran nucleoside analog of Formula X:

Bx is a heterocyclic base moiety;

T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to an oligomeric compound or oligonucleotide and the other of T₃ and T₄ is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and

one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula X are provided wherein qₘ, qₙ, qₚ, qᵣ, qₛ, qₜ and qᵤ are each H. In certain embodiments, at least one of qₘ, qₙ, qₚ, qᵣ, qₛ, qₜ and qᵤ is other than H. In certain embodiments, at least one of qₘ, qₙ, qₚ, qᵣ, qₛ, qₜ and qᵤ is methyl. In certain embodiments, THP nucleosides of Formula X are provided wherein one of R₁ and R₂ is F. In certain embodiments, R₁ is fluoro and R₂ is H; R₁ is methoxy and R₂ is H, and R₁ is methoxyethoxy and R₂ is H.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂CH₃ group at the 2′ position of the sugar ring.

As used herein, the term “adenine analogue” means a chemically-modified purine nucleobase that, when incorporated into an oligomer, is capable with forming a Watson-Crick base pair with either a thymidine or uracil of a complementary strand of RNA or DNA.

As used herein, the term “uracil analogue” means a chemically-modified pyrimidine nucleobase that, when incorporated into an oligomer, is capable with forming a Watson-Crick base pair with either a adenine of a complementary strand of RNA or DNA.

As used herein, the term “thymine analogue” means a chemically-modified adenine nucleobase that, when incorporated into an oligomer, is capable with forming a Watson-Crick base pair with an adenine of a complementary strand of RNA or DNA.

As used herein, the term “cytosine analogue” means a chemically-modified pyrimidine nucleobase that, when incorporated into an oligomer, is capable with forming a Watson-Crick base pair with a guanine of a complementary strand of RNA or DNA. For example, cytosine analogue can be a 5-methylcytosine.

As used herein, the term “guanine analogue” means a chemically-modified purine nucleobase that, when incorporated into an oligomer, is capable with forming a Watson-Crick base pair with a cytosine of a complementary strand of RNA or DNA.

As used herein, the term “guanosine” refers to a nucleoside or sugar-modified nucleoside comprising a guanine or guanine analog nucleobase.

As used herein, the term “uridine” refers to a nucleoside or sugar-modified nucleoside comprising a uracil or uracil analog nucleobase.

As used herein, the term “thymidine” refers to a nucleoside or sugar-modified nucleoside comprising a thymine or thymine analog nucleobase.

As used herein, the term “cytidine” refers to a nucleoside or sugar-modified nucleoside comprising a cytosine or cytosine analog nucleobase.

As used herein, the term “adenosine” refers to a nucleoside or sugar-modified nucleoside comprising an adenine or adenine analog nucleobase.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein “oligonucleoside” refers to an oligonucleotide in which none of the internucleoside linkages contains a phosphorus atom. As used herein, oligonucleotides include oligonucleosides.

As used herein, “modified oligonucleotide” or “chemically-modified oligonucleotide” refers to an oligonucleotide comprising at least one modified sugar, a modified nucleobase and/or a modified internucleoside linkage.

As used herein “internucleoside linkage” refers to a covalent linkage between adjacent nucleosides.

As used herein “naturally occurring internucleoside linkage” refers to a 3′ to 5′ phosphodiester linkage.

As used herein, “modified internucleoside linkage” refers to any internucleoside linkage other than a naturally occurring internucleoside linkage.

As used herein, “oligomeric compound” refers to a polymeric structure comprising two or more sub-structures. In certain embodiments, an oligomeric compound is an oligonucleotide. In certain embodiments, an oligomeric compound is a single-stranded oligonucleotide. In certain embodiments, an oligomeric compound is a double-stranded duplex comprising two oligonucleotides. In certain embodiments, an oligomeric compound is a single-stranded or double-stranded oligonucleotide comprising one or more conjugate groups and/or terminal groups.

As used herein, “conjugate” refers to an atom or group of atoms bound to an oligonucleotide or oligomeric compound. In general, conjugate groups modify one or more properties of the compound to which they are attached, including, but not limited to pharmakodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleosides of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “antisense compound” refers to an oligomeric compound, at least a portion of which is at least partially complementary to a target nucleic acid to which it hybridizes and modulates the activity, processing or expression of said target nucleic acid.

As used herein, “expression” refers to the process by which a gene ultimately results in a protein. Expression includes, but is not limited to, transcription, splicing, post-transcriptional modification, and translation.

As used herein, “antisense oligonucleotide” refers to an antisense compound that is an oligonucleotide.

As used herein, “antisense activity” refers to any detectable and/or measurable activity attributable to the hybridization of an antisense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. In certain embodiments, antisense activity is assessed by observing a phenotypic change in a cell or animal.

As used herein, “detecting” or “measuring” in connection with an activity, response, or effect indicate that a test for detecting or measuring such activity, response, or effect is performed. Such detection and/or measuring may include values of zero. Thus, if a test for detection or measuring results in a finding of no activity (activity of zero), the step of detecting or measuring the activity has nevertheless been performed. For example, in certain embodiments, the present invention provides methods that comprise steps of detecting antisense activity, detecting toxicity, and/or measuring a marker of toxicity. Any such step may include values of zero.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is mRNA, pre-mRNA, non-coding RNA, pri-microRNA, pre-microRNA, mature microRNA, promoter-directed RNA, or natural antisense transcripts. For example, the target nucleic acid can be a cellular gene (or mRNA transcribed from the gene) whose expression is associated with a particular disorder or disease state, or a nucleic acid molecule from an infectious agent. In certain embodiments, target nucleic acid is a viral or bacterial nucleic acid.

As used herein, “target mRNA” refers to a pre-selected RNA molecule that encodes a protein.

As used herein, “target pdRNA” refers to refers to a pre-selected RNA molecule that interacts with one or more promoter to modulate transcription.

As used herein, “targeting” or “targeted to” refers to the association of an antisense compound to a particular target nucleic acid molecule or a particular region of nucleotides within a target nucleic acid molecule. An antisense compound targets a target nucleic acid if it is sufficiently complementary to the target nucleic acid to allow hybridization under physiological conditions.

As used herein, “target site” refers to a region of a target nucleic acid that is bound by an antisense compound. In certain embodiments, a target site is at least partially within the 3′ untranslated region of an RNA molecule. In certain embodiments, a target site is at least partially within the 5′ untranslated region of an RNA molecule. In certain embodiments, a target site is at least partially within the coding region of an RNA molecule. In certain embodiments, a target site is at least partially within an exon of an RNA molecule. In certain embodiments, a target site is at least partially within an intron of an RNA molecule. In certain embodiments, a target site is at least partially within a microRNA target site of an RNA molecule. In certain embodiments, a target site is at least partially within a repeat region of an RNA molecule.

As used herein, “target protein” refers to a protein, the expression of which is modulated by an antisense compound. In certain embodiments, a target protein is encoded by a target nucleic acid. In certain embodiments, expression of a target protein is otherwise influenced by a target nucleic acid.

As used herein, “complementarity” in reference to nucleobases refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary”” in reference to nucleobases refers to a pair of nucleobases that do not form hydrogen bonds with one another or otherwise support hybridization.

As used herein, “complementary” in reference to linked nucleosides, oligonucleotides, or nucleic acids, refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

As used herein, “hybridization” refers to the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methylcytosine. Hybridization can occur under varying circumstances.

As used herein, “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “overall identity” refers to the nucleobase identity of an oligomeric compound relative to a particular nucleic acid or portion thereof, over the length of the oligomeric compound.

As used herein, “modulation” refers to a perturbation of amount or quality of a function or activity when compared to the function or activity prior to modulation. For example, modulation includes the change, either an increase (stimulation or induction) or a decrease (inhibition or reduction) in gene expression. As a further example, modulation of expression can include perturbing splice site selection of pre-mRNA processing, resulting in a change in the amount of a particular splice-variant present compared to conditions that were not perturbed. As a further example, modulation includes perturbing translation of a protein.

As used herein, “motif” refers to a pattern of modifications in an oligomeric compound or a region thereof. Motifs may be defined by modifications at certain nucleosides and/or at certain linking groups of an oligomeric compound.

As used herein, “nucleoside motif” refers to a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “linkage motif” refers to a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “the same modifications” refer to modifications relative to naturally occurring molecules that are the same as one another, including absence of modifications. Thus, for example, two unmodified DNA nucleoside have “the same modification,” even though the DNA nucleoside is unmodified.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “separate regions” refers to a portion of an oligomeric compound wherein the nucleosides and internucleoside linkages within the region all comprise the same modifications; and the nucleosides and/or the internucleoside linkages of any neighboring portions include at least one different modification.

As used herein, “pharmaceutically acceptable salts” refers to salts of active compounds that retain the desired biological activity of the active compound and do not impart undesired toxicological effects thereto.

As used herein, “cap structure” or “terminal cap moiety” refers to chemical modifications incorporated at either terminus of an antisense compound.

As used herein, the term “independently” means that each occurrence of a repetitive variable within a claimed oligonucleotide is selected independent of one another. For example, each repetitive variable can be selected so that (i) each of the repetitive variables are the same, (ii) two or more are the same, or (iii) each of the repetitive variables can be different.

### General Chemistry Definitions

As used herein, “alkyl,” refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like.

Alkyl groups typically include from 1 to about 24 carbon atoms, more typically from 1 to about 12 carbon atoms (C₁-C₁₂ alkyl) with from 1 to about 6 carbon atoms (C₁-C₆ alkyl) being more preferred. The term “lower alkyl” as used herein includes from 1 to about 6 carbon atoms (C₁-C₆ alkyl). Alkyl groups as used herein may optionally include one or more further substituent groups. Herein, the term “alkyl” without indication of number of carbon atoms means an alkyl having 1 to about 12 carbon atoms (C₁-C₁₂ alkyl).

As used herein, “alkenyl,” refers to a straight or branched hydrocarbon chain radical containing up to twenty four carbon atoms and having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “aminoalkyl” refers to an amino substituted alkyl radical. This term is meant to include C₁-C₁₂ alkyl groups having an amino substituent at any position and wherein the alkyl group attaches the aminoalkyl group to the parent molecule. The alkyl and/or amino portions of the aminoalkyl group can be further substituted with substituent groups.

As used herein, “aliphatic,” refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines. Aliphatic groups as used herein may optionally include further substituent groups.

As used herein, “alicyclic” or “alicyclyl” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “alkoxy,” refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “halo” and “halogen,” refer to an atom selected from fluorine, chlorine, bromine and iodine.

As used herein, “aryl” and “aromatic,” refer to a mono- or polycyclic carbocyclic ring system radicals having one or more aromatic rings. Examples of aryl groups include, but are not limited to, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “aralkyl” and “arylalkyl,” refer to a radical formed between an alkyl group and an aryl group wherein the alkyl group is used to attach the aralkyl group to a parent molecule. Examples include, but are not limited to, benzyl, phenethyl and the like. Aralkyl groups as used herein may optionally include further substituent groups attached to the alkyl, the aryl or both groups that form the radical group.

As used herein, “heterocyclic radical” refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain at least one heteroatom and the other rings can contain one or more heteroatoms or optionally contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substituent groups.

As used herein, “heteroaryl,” and “heteroaromatic,” refer to a radical comprising a mono- or poly-cyclic aromatic ring, ring system or fused ring system wherein at least one of the rings is aromatic and includes one or more heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals can be attached to a parent molecule directly or through a linking moiety such as an aliphatic group or hetero atom. Heteroaryl groups as used herein may optionally include further substituent groups.

As used herein, “heteroarylalkyl,” refers to a heteroaryl group as previously defined having an alky radical that can attach the heteroarylalkyl group to a parent molecule. Examples include, but are not limited to, pyridinylmethyl, pyrimidinylethyl, napthyridinylpropyl and the like. Heteroarylalkyl groups as used herein may optionally include further substituent groups on one or both of the heteroaryl or alkyl portions.

As used herein, “mono or poly cyclic structure” refers to any ring systems that are single or polycyclic having rings that are fused or linked and is meant to be inclusive of single and mixed ring systems individually selected from aliphatic, alicyclic, aryl, heteroaryl, aralkyl, arylalkyl, heterocyclic, heteroaryl, heteroaromatic, heteroarylalkyl. Such mono and poly cyclic structures can contain rings that are uniform or have varying degrees of saturation including fully saturated, partially saturated or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or poly cyclic structures can be further substituted with substituent groups such as for example phthalimide which has two ═O groups attached to one of the rings. In another aspect, mono or poly cyclic structures can be attached to a parent molecule directly through a ring atom, through a substituent group or a bifunctional linking moiety.

As used herein, “acyl,” refers to a radical formed by removal of a hydroxyl group from an organic acid and has the general formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “hydrocarbyl” refers to any group comprising C, O and H. Included are straight, branched and cyclic groups having any degree of saturation. Such hydrocarbyl groups can include one or more heteroatoms selected from N, O and S and can be further mono or poly substituted with one or more substituent groups.

As used herein, “substituent” and “substituent group,” include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to a parent compound.

Unless otherwise indicated, the term substituted or “optionally substituted” refers to the following substituents: halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxo (—O—Rₐₐ), aryl, aralkyl, heterocyclic, heteroaryl, heteroarylalkyl, amino (—NR_{bb}R_{cc}), imino(═NR_{bb}), amido (—C(O)N—R_{bb}R_{cc} or —N(R_{bb})C(O)Rₐₐ), azido (—N₃), nitro (—NO₂), cyano (—CN), carbamido (—OC(O)NR_{bb}R_{cc} or —N(R_{bb})C(O)ORₐₐ), ureido (—N(R_{bb})C(O)NR_{bb}R_{cc}), thioureido (—N(R_{bb})C(S)NR_{bb}R_{cc}), guanidinyl (—N(R_{bb})C(═NR_{bb})NR_{bb}R_{cc}), amidinyl (—C(═NR_{bb})NR_{bb}R_{cc} or —N(R_{bb})C(NR_{bb})Rₐₐ), thiol (—SR_{bb}), sulfinyl (—S(O)R_{bb}), sulfonyl (—S(O)₂R_{bb}), sulfonamidyl (—S(O)₂NR_{bb}R_{cc} or —N(R_{bb})—S(O)₂R_{bb}) and conjugate groups. Wherein each Rₐₐ, R_{bb} and R_{cc} is, independently, H, an optionally linked chemical functional group or a further substituent group with a preferred list including without limitation H, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by way of example and not limitation, physical properties such as molecular weight, solubility or log P, application properties such as activity against the intended target and practical properties such as ease of synthesis.

Recursive substituents are an intended aspect of the invention. One of ordinary skill in the art of medicinal and organic chemistry understands the versatility of such substituents. To the degree that recursive substituents are present in a claim of the invention, the total number will be determined as set forth above.

The terms “stable compound” and “stable structure” as used herein are meant to indicate a compound that is sufficiently robust to survive isolation to a useful degree of purity from a reaction mixture, and formulation into an efficacious therapeutic agent. Only stable compounds are contemplated herein.

As used herein, a zero (0) in a range indicating number of a particular unit means that the unit may be absent. For example, an oligomeric compound comprising 0-2 regions of a particular motif means that the oligomeric compound may comprise one or two such regions having the particular motif, or the oligomeric compound may not have any regions having the particular motif. In instances where an internal portion of a molecule is absent, the portions flanking the absent portion are bound directly to one another. Likewise, the term “none” as used herein, indicates that a certain feature is not present.

As used herein, “analogue” or “derivative” means either a compound or moiety similar in structure but different in respect to elemental composition from the parent compound regardless of how the compound is made. For example, an analogue or derivative compound does not need to be made from the parent compound as a chemical starting material.

### Certain Nucleobases

In certain embodiments, nucleosides of the present invention comprise unmodified nucleobases. In certain embodiments, nucleosides of the present invention comprise modified nucleobases.

In certain embodiments, nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Certain modified nucleobases are disclosed in, for example, Swayze, E. E. and Bhat, B., The Medicinal Chemistry of Oligonucleotides in ANTISENSE DRUG TECHNOLOGY, Chapter 6, pages 143-182 (Crooke, S. T., ed., 2008); U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2 aminopropyladenine, 5-propynyluracil and 5-propynylcytosine.

In certain embodiments, nucleobases comprise polycyclic heterocyclic compounds in place of one or more heterocyclic base moieties of a nucleobase. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs.

Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). When incorporated into oligonucleotides, these base modifications have been shown to hybridize with complementary guanine and the latter was also shown to hybridize with adenine and to enhance helical thermal stability by extended stacking interactions (also see U.S. Patent Application Publication 20030207804 and U.S. Patent Application Publication 20030175906, both of which are incorporated herein by reference in their entirety).

Helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Tricyclic heterocyclic compounds and methods of using them that are amenable to the present invention are disclosed in U.S. Pat. No. 6,028,183, and U.S. Pat. No. 6,007,992, the contents of both are incorporated herein in their entirety.

The enhanced binding affinity of the phenoxazine derivatives together with their sequence specificity makes them valuable nucleobase analogs for the development of more potent antisense-based drugs. The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

Modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Patent Application Publication 20030158403, each of which is incorporated herein by reference in its entirety.

### Sugar-Modified Nucleosides

RNA duplexes exist in what has been termed “A Form” geometry while DNA duplexes exist in “B Form” geometry. In general, RNA:RNA duplexes are more stable, or have higher melting temperatures (Tₘ) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.).

The relative ability of a chemically-modified oligomeric compound to bind to comple-mentary nucleic acid strands, as compared to natural oligonucleotides, is measured by obtaining the melting temperature of a hybridization complex of said chemically-modified oligomeric compound with its complementary unmodified target nucleic acid. The melting temperature (Tₘ), a characteristic physical property of double helixes, denotes the temperature in degrees centigrade at which 50% helical versus coiled (unhybridized) forms are present. Tₘ (also commonly referred to as binding affinity) is measured by using the UV spectrum to determine the formation and breakdown (melting) of hybridization. Base stacking, which occurs during hybridization, is accompanied by a reduction in UV absorption (hypochromicity). Consequently a reduction in UV absorption indicates a higher Tₘ.

It is known in the art that the relative duplex stability of an antisense compound:RNA target duplex can be modulated through incorporation of chemically-modified nucleosides into the antisense compound. Sugar-modified nucleosides have provided the most efficient means of modulating the Tₘ of an antisense compound with its target RNA. Sugar-modified nucleosides that increase the population of or lock the sugar in the C3′-endo (Northern, RNA-like sugar pucker) configuration have predominantly provided a per modification Tₘ increase for antisense compounds toward a complementary RNA target. Sugar-modified nucleosides that increase the population of or lock the sugar in the C2′-endo (Southern, DNA-like sugar pucker) configuration predominantly provide a per modification Tₘ decrease for antisense compounds toward a complementary RNA target. The sugar pucker of a given sugar-modified nucleoside is not the only factor that dictates the ability of the nucleoside to increase or decrease an antisense compound's Tₘ toward complementary RNA. For example, the sugar-modified nucleoside tricycloDNA is predominantly in the C2′-endo conformation, however it imparts a 1.9 to 3° C. per modification increase in Tₘ toward a complementary RNA. Another example of a sugar-modified high-affinity nucleoside that does not adopt the C3′-endo conformation is α-L-LNA (described in more detail herein).

### Certain Oligonucleotides

In certain embodiments, the present invention provides modified oligonucleotides. In certain embodiments, modified oligonucleotides of the present invention comprise modified nucleosides. In certain embodiments, modified oligonucleotides of the present invention comprise modified internucleoside linkages. In certain embodiments, modified oligonucleotides of the present invention comprise modified nucleosides and modified internucleoside linkages.

In certain embodiments, the invention provides mutant selective compounds, which have a greater effect on a mutant nucleic acid than on the corresponding wild-type nucleic acid. In certain embodiment, the effect of a mutant selective compound on the mutant nucleic acid is 2, 3, 4, 5, 6, 7, 8, 9, 10, 15, 20, or 100 times greater than the effect of the mutant selective compound on the corresponding wild-type nucleic acid. In certain embodiments, such selectivity results from greater affinity of the mutant selective compound for the mutant nucleic acid than for the corresponding wild type nucleic acid. In certain embodiments, selectivity results from a difference in the structure of the mutant compared to the wild-type nucleic acid. In certain embodiments, selectivity results from differences in processing or sub-cellular distribution of the mutant and wild-type nucleic acids. In certain embodiments, some selectivity may be attributable to the presence of additional target sites in a mutant nucleic acid compared to the wild-type nucleic acid. For example, in certain embodiments, a target mutant allele comprises an expanded repeat region comprising additional copies of a target sequence, while the wild-type allele has fewer copies of the repeat and, thus, fewer sites for hybridization of an antisense compound targeting the repeat region. In certain embodiments, a mutant selective compound has selectivity equal to or greater than the selectivity predicted by the increased number of target sites. In certain embodiments, a mutant selective compound has selectivity greater than the selectivity predicted by the increased number of target sites. In certain embodiments, the ratio of inhibition of a mutant allele to a wild type allele is equal to or greater than the ratio of the number of repeats in the mutant allele to the wild type allele. In certain embodiments, the ratio of inhibition of a mutant allele to a wild type allele is greater than the ratio of the number of repeats in the mutant allele to the wild type allele.

### Certain Internucleoside Linkages

In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligonucleotides having non-phosphorus internucleoside linking groups may be referred to as oligonucleosides. Modified linkages, compared to natural phosphodiester linkages, can be used to alter, typically increase, nuclease resistance of the oligomeric compound. In certain embodiments, internucleoside linkages having a chiral atom can be prepared a racemic mixture, as separate enantomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantomers, diastereomers, and other stereoisomeric configurations that may be defined, in terms of absolute stereochemistry, as (R) or (S), α or β such as for sugar anomers, or as (D) or (L) such as for amino acids et al. Included in the antisense compounds provided herein are all such possible isomers, as well as their racemic and optically pure forms.

As used herein the term “internucleoside linkage” or “internucleoside linking group” is meant to include all manner of internucleoside linking groups known in the art including but not limited to, phosphorus containing internucleoside linking groups such as phosphodiester and phosphorothioate, and non-phosphorus containing internucleoside linking groups such as formacetyl and methyleneimino. Internucleoside linkages also includes neutral non-ionic internucleoside linkages such as amide-3 (3′-CH₂—C(═O)—N(H)-5′), amide-4 (3′-CH₂—N(H)—C(═OO)-5′) and methylphosphonate wherein a phosphorus atom is not always present.

As used herein the phrase “neutral internucleoside linkage” is intended to include internucleoside linkages that are non-ionic. Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

The internucleotide linkage found in native nucleic acids is a phosphodiester linkage. This linkage has not been the linkage of choice for synthetic oligonucleotides that are for the most part targeted to a portion of a nucleic acid such as mRNA because of stability problems e.g. degradation by nucleases. Preferred internucleotide linkages or internucleoside linkages as is the case for non phospate ester type linkages include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified internucleoside linkages that do not include a phosphorus atom therein include short chain alkyl or cycloalkyl internucleoside linkages, mixed heteroatom and alkyl or cycloalkyl internucleoside linkages, or one or more short chain heteroatomic or heterocyclic internucleoside linkages. These include siloxane, sulfide, sulfoxide, sulfone, formacetyl, thioformacetyl, methylene formacetyl, thioformacetyl, alkenyl, sulfamate, methyleneimino, methylenehydrazino, sulfonate, sulfonamide, amide and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Most preferred embodiments of the invention are oligomeric compounds with phosphorothioate internucleoside linkages and oligomeric compounds with heteroatom internucleoside linkages, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P(═O)(OH)—O—CH₂—] of the above referenced U.S. Pat. No. 5,489,677, and the amide internucleoside linkages of the above referenced U.S. Pat. No. 5,602,240. Also preferred are oligonucleotides having morpholino backbone structures of the above-referenced U.S. Pat. No. 5,034,506.

### 3′ and 5′-Base Modifications and Conjugates

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present invention involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-5-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned, and each of which is herein incorporated by reference.

### Oligonucleotide Synthesis

Commercially available equipment routinely used for the support media based synthesis of oligomeric compounds and related compounds is sold by several vendors including, for example, Applied Biosystems (Foster City, Calif.), General Electric, as well as others. Suitable solid phase techniques, including automated synthesis techniques, are described in Scozzari and Capaldi, “Oligonucleotide Manufacturing and Analysic Processes for 2′-O-(2-methoxyethyl-Modified Oligonucleotides” in Crooke, S T (ed.) ANTISENSE THERAPEUTICS (2008).

### Certain Uses

In certain embodiments, described herein is use of a chemically-modified oligonucleotide 13 to 22 nucleobases in length and having a nucleobase sequence comprising SEQ ID NO.: 2 [TGCTGCTGCTG] and 100% complementary within a repeat region of a CAG nucleotide repeat containing RNA, wherein:
a. each T is independently a uridine or thymidine nucleoside and each comprising an independently selected high-affinity sugar modification;
b. each non-terminal G is a guanosine nucleoside comprising a 2′-deoxyribose sugar;
c. each non-terminal C is a cytidine nucleoside comprises a 2′-deoxyribose sugar; and
   wherein one or both of the 5′ or 3′ terminal nucleosides of the chemically-modified oligonucleotide independently comprises one or more nuclease-resistant modification; for the treatment of a disease associated with a CAG nucleotide repeat-containing RNA.

In certain uses, the disease is any of Atrophin 1, Huntington's Disease, Huntington disease-like 2 (HDL2), spinal and bulbar muscular atrophy, Kennedy disease, spinocerebellar ataxia 1, spinocerebellar ataxia 12, spinocerebellar ataxia 17, Huntington disease-like 4 (HDL4), spinocerebellar ataxia 2, spinocerebellar ataxia 3, Machado-Joseph disease, spinocerebellar ataxia 6, and spinocerebellar ataxia 7.

In certain embodiments, described herein is use of a chemically-modified oligonucleotide 13 to 22 nucleobases in length comprising a nucleobase sequence of SEQ ID NO.: 2 [TGCTGCTGCTG] which is 100% complementary within a repeat region of a CAG nucleotide repeat containing RNA, wherein:
a. each G is a guanosine nucleoside independently comprises a high affinity sugar modification;
b. each non-terminal T is independently a uridine or thymidine nucleoside comprising a 2′-deoxyribose sugar;
c. each terminal T is independently a uridine or thymidine nucleoside comprising a 2′deoxyribose sugar or a nuclease resistant modification;
d. each non-terminal C is a cytidine nucleoside comprising a 2′-deoxyribose sugar; and
e. each terminal C is a cytidine nucleoside comprising either a 2′deoxyribose sugar or a nuclease resistant modification
   for the treatment of a disease associated with a CAG nucleotide repeat-containing RNA.

In certain uses, the disease is any of Atrophin 1, Huntington's Disease, Huntington disease-like 2 (HDL2), spinal and bulbar muscular atrophy, Kennedy disease, spinocerebellar ataxia 1, spinocerebellar ataxia 12, spinocerebellar ataxia 17, Huntington disease-like 4 (HDL4), spinocerebellar ataxia 2, spinocerebellar ataxia 3, Machado-Joseph disease, spinocerebellar ataxia 6, and spinocerebellar ataxia 7.

In certain embodiments, described herein is use of a chemically-modified oligonucleotide 13 to 22 nucleobases in length and having a nucleobase sequence comprising SEQ ID NO.: 4 [AGCAGCAGCAG] and 100% complementary within a repeat region of a CUG nucleotide repeat containing RNA, wherein:
a. each A is independently a adenosine nucleoside, each comprising an independently selected high-affinity sugar modification;
b. each non-terminal G is a guanosine nucleoside comprising a 2′-deoxyribose sugar;
c. each terminal G is a guanosine nucleoside comprising independently a 2′-deoxyribose sugar and/or a nuclease resistant modification;
d. each non-terminal C is a cytidine nucleoside comprises a 2′-deoxyribose sugar; and
e. each terminal C is a cytidine nucleoside comprising independently a 2′-deoxyribose sugar and/or a nuclease resistant modification.

for the treatment of a disease associated with a CUG nucleotide repeat-containing RNA.

In certain uses, the disease is any of Huntington disease-like 2 (HDL2), Myotonic Dystrophy (DM1), or spinocerebellar ataxia 8.

In certain embodiments, described herein is use of a chemically-modified oligonucleotide 13 to 22 nucleobases in length comprising a nucleobase sequence of SEQ ID NO.: 4 [AGCAGCAGCAG] which is 100% complementary within a repeat region of a CUG nucleotide repeat containing RNA, wherein:
a. each G is a guanosine nucleoside independently comprising a high affinity sugar modification;
b. each non-terminal A is independently an adenosine nucleoside comprising a 2′-deoxyribose sugar;
c. each terminal A is independently an adenosine nucleoside comprising a 2′deoxyribose sugar or a nuclease resistant modification;
d. each non-terminal C is a cytidine nucleoside comprising a 2′-deoxyribose sugar; and
e. each terminal C is a cytidine nucleoside comprising either a 2′deoxyribose sugar or a nuclease resistant modification;
   for the treatment of a disease associated with a CUG nucleotide repeat-containing RNA

In certain uses, the disease is any of Huntington disease-like 2 (HDL2), Myotonic Dystrophy (DM1), or spinocerebellar ataxia 8.

### Administration

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump. In certain embodiments, parenteral administration is by injection.

In certain embodiments, compounds and compositions are delivered to the CNS. In certain embodiments, compounds and compositions are delivered to the cerebrospinal fluid. In certain embodiments, compounds and compositions are administered to the brain parenchyma. In certain embodiments, compounds and compositions are delivered to an animal by intrathecal administration, or intracerebroventricular administration. Broad distribution of compounds and compositions, described herein, within the central nervous system may be achieved with intraparenchymal administration, intrathecal administration, or intracerebroventricular administration.

In certain embodiments, parenteral administration is by injection. The injection may be delivered with a syringe or a pump. In certain embodiments, the injection is a bolus injection. In certain embodiments, the injection is administered directly to a tissue, such as striatum, caudate, cortex, hippocampus and cerebellum.

In certain embodiments, delivery of a compound or composition described herein can affect the pharmacokinetic profile of the compound or composition. In certain embodiments, injection of a compound or composition described herein, to a targeted tissue improves the pharmacokinetic profile of the compound or composition as compared to infusion of the compound or composition. In a certain embodiment, the injection of a compound or composition improves potency compared to broad diffusion, requiring less of the compound or composition to achieve similar pharmacology. In certain embodiments, similar pharmacology refers to the amount of time that a target mRNA and/or target protein is down-regulated (e.g. duration of action). In certain embodiments, methods of specifically localizing a pharmaceutical agent, such as by bolus injection, decreases median effective concentration (EC50) by a factor of about 50 (e.g. 50 fold less concentration in tissue is required to achieve the same or similar pharmacodynamic effect). In certain embodiments, methods of specifically localizing a pharmaceutical agent, such as by bolus injection, decreases median effective concentration (EC50) by a factor of 20, 25, 30, 35, 40, 45 or 50. In certain embodiments, the pharmaceutical agent in an antisense compound as further described herein. In certain enbodiments, the targeted tissue is brain tissue. In certain enbodiments the targeted tissue is striatal tissue. In certain embodiments, decreasing EC50 is desirable because it reduces the dose required to achieve a pharmacological result in a patient in need thereof.

In certain embodiments, delivery of a compound or composition, as described herein, to the CNS results in 47% down-regulation of a target mRNA and/or target protein for at least 91 days. In certain embodiments, delivery of a compound or composition results in at least 25%, at least 30%, at least 35%, at least 40%, at least 45%, at least 50%, at least 55%, at least 60%, at least 65%, at least 70%, or at least 75% down-regulation of a target mRNA and/or target protein for at least 20 days, at least 30 days, at least 40 days, at least 50 days, at least 60 days, at least 70 days, at least 80 days, at least 85 days, at least 90 days, at least 95 days, at least 100 days, at least 110 days, at least 120 days. In certain embodiments, delivery to the CNS is by intraparenchymal administration, intrathecal administration, or intracerebroventricular administration.

In certain embodiments, an antisense oligonucleotide is delivered by injection or infusion once every month, every two months, every 90 days, every 3 months, every 6 months, twice a year or once a year.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

Throughout herein below the following notations mean the following: ‘L’=LNA; ‘E’ or ‘k’=cEt; italicized bases have 2′-O-methoxyethyl ribose modifications; ‘d’=deoxyribose; ‘s’=phosphorothioate; ‘l’=cLNA or carbacyclic-LNA (except Table 17 where ‘l’=LNA); and mC=5-methylcytosine.

### Example 1

### Effect of LNA-Modified Oligonucleotides, Targeting Human Huntingtin (htt) mRNA, on Huntingtin (Htt) Protein

Antisense oligonucleotides targeted to the CAG repeat sequence of mutant huntingtin mRNA having LNA modifications were tested for their effect on Htt protein levels in vitro. The GM04281 fibroblast cell line (Coriell Institute for Medical Research, NJ, USA) containing 69 CAG repeats in the mutant htt allele and 17 CAG repeats in the wild-type allele, was used in this assay. Cells were cultured at a density of 60,000 cells per well in 6-well plates and were transfected using Lipofectamine™ RNAiMAX reagent (Invitrogen, CA) with 100 nM antisense oligonucleotide for 24 hours. The wells were then aspirated and fresh culture medium was added to each well.

After a post-transfection period of 4 days, the cells were harvested with trypsin solution (0.05% Trypsin-EDTA, Invitrogen) and lysed. The protein concentration in each sample was quantified with the micro-bicinchoninic acid (micro-BCA) assay (Thermo Scientific). An SDS-PAGE gel (Bio-Rad) was used to separate wild-type and mutant Htt proteins. Gels were run at 80 V for 15 min followed by 110 V for 5 hr. The electrophoresis apparatus was placed in an ice-water bath to prevent overheating. In parallel with analysis for Htt expression, portions of each protein lysate sample were also analyzed for β-actin expression by SDS-PAGE to ensure that there had been equal protein loading of each sample.

After electrophoresis, proteins in the gel were transferred to a nitrocellulose membrane (Hybond-C Extra; GE Healthcare Bio-Sciences). Primary antibodies specific for Htt (MAB2166, Chemicon) and β-actin (Sigma) protein were used at 1:10,000 dilutions. HRP-conjugated anti-mouse secondary antibody (1:10,000, Jackson ImmunoResearch Laboratories) was used for visualizing proteins using SuperSignal West Pico Chemiluminescent Substrate (Thermo Scientific). Protein bands were quantified using ImageJ software. The percentage inhibition was calculated relative to the negative control sample and presented in Table 1. The comparative percent inhibitions of the wild-type Htt protein and the mutant Htt protein are also presented. The Tₘ value for each oligonucleotide, determined by differential scanning calorimetry (DSC) is also shown.

The antisense oligonucleotides utilized in the assay are described in Table 1. The antisense oligonucleotides were obtained from either Sigma Aldrich or ISIS Pharmaceuticals. Of the antisense oligonucleotides presented in Table 1, DNA22 is an unmodified oligonucleotide (DNA nucleosides with phosphodiester linkages). The negative control is a scrambled oligonucleotide sequence. The LNA modifications in each oligonucleotide are indicated by the subscript ‘L’ after each base.

TABLE 1
Effect of LNA-modified antisense oligonucleotides on
wild-type and mutant Htt protein

| | | | | % inhibition | | SEQ |
|---|---|---|---|---|---|---|
| Oligo | | | Tₘ | wild- | | ID |
| ID | Sequence | Length | (° C.) | type | Mutant | NO |
| DNA22 | GCTGCTGCTGCTGCTGCTGCTG | 22 | 77 | 0 | 0 | 7 |
| (−) | GCT_{L}ATA_{L}CCA_{L}GCG_{L}TCG_{L}TCA_{L}T | 19 | 0 | 0 | 0 | 8 |
| control | | | | | | |
| LNA(T) | GCT_{L}GCT_{L}GCT_{L}GCT_{L}GCT_{L}GCT_{L}G | 19 | 97 | 18 | 62 | 9 |
| LNA(G) | G_{L}CTG_{L}CTG_{L}CTG_{L}CTG_{L}CTG_{L}CTG_{L} | 19 | 96 | 19 | 64 | 9 |
| LNA(C) | GC_{L}TGC_{L}TGC_{L}TGC_{L}TGC_{L}TGC_{L}TG | 19 | 97 | 2 | 32 | 9 |
| LNA(T) + 1 | CT_{L}GCT_{L}GCT_{L}GCT_{L}GCT_{L}GCT_{L}GC | 19 | 96 | 18 | 58 | 10 |
| LNA(G) + 1 | CTG_{L}CTG_{L}CTG_{L}CTG_{L}CTG_{L}CTG_{L}C | 19 | 94 | 15 | 52 | 10 |
| LNA(T) + 2 | T_{L}GCT_{L}GCT_{L}GCT_{L}GCT_{L}GCT_{L}GCT_{L} | 19 | 96 | 19 | 84 | 11 |
| LNA(C) + 2 | TGC_{L}TGC_{L}TGC_{L}TGC_{L}TGC_{L}TGC_{L}T | 19 | 96 | 10 | 32 | 1 |
| LNA (T)22 | GCT_{L}GCT_{L}GCT_{L}GCT_{L}GCT_{L}GCT_{L}GCT_{L}G | 22 | 99 | 34 | 71 | 7 |
| LNA(T)16 | GCT_{L}GCT_{L}GCT_{L}GCT_{L}GCT_{L}G | 16 | 92 | 18 | 31 | 12 |
| LNA(T)13 | GCT_{L}GCT_{L}GCT_{L}GCT_{L}G | 13 | 88 | 10 | 27 | 13 |
| LNA-gap | G_{L}C_{L}T_{L}G_{L}CTGCTGCTGCTG_{L}C_{L}T_{L}G_{L} | 19 | 95 | 44 | 74 | 9 |

Several of the oligonucleotides reduced nucleotide repeat-containing RNA more than they reduced the corresponding wild-type.

### Example 2

### In Vitro Dose-Dependent Effect of LNA-Modified Nucleotides on Human Htt Protein

Antisense oligonucleotides from Example 1 (see Table 1 for description of chemical modifications) were tested at various doses in patient fibroblast cells. GM04281 fibroblast cells were plated at a density of 60,000 cells per well in 6-well plates and transfected using Lipofectamine™ RNAiMAX reagent (Invitrogen, CA) reagent with increasing concentrations of antisense oligonucleotide for 24 hours, as specified in Tables 2 and 3. The cell samples were processed for protein analysis utilizing the procedure outlined in Example 1.

Results are presented in Tables 2 and 3 as percent inhibition of wild-type and mutant Htt protein, relative to untreated control cells. The data presented is an average of several independent assays performed with each antisense oligonucleotide. As illustrated in Table 2, Htt mutant protein levels were reduced in a dose-dependent manner in antisense oligonucleotide treated cells. The IC₅₀ (nM) values for each oligonucleotide for inhibition of the mutant protein and wild-type protein is also shown and indicates that each oligonucleotide preferentially targets the mutant htt mRNA compared to the wild-type. The oligonucleotides listed in Table 3 demonstrate low in vitro potency and or little or no preferential lowering of the mutant mRNA compared to the wild-type.

TABLE 2
Dose-dependent effect of LNA-modified oligonucleotides on wild-type versus mutant Htt protein

| | SEQ | | | | | | | | |
|---|---|---|---|---|---|---|---|---|---|
| | ID | Htt | | | | | | | IC₅₀ |
| Oligo ID | NO | protein | 3.12 nM | 6.25 nM | 12.5 nM | 25 nM | 50 nM | 100 nM | (nM) |
| LNA(T) | 9 | wild-type | 2 | 3 | 9 | 12 | 15 | 21 | >100 |
| | | mutant | 5 | 15 | 28 | 42 | 54 | 68 | 39.8 |
| LNA(G) | 9 | wild-type | 0 | 10 | 15 | 25 | 24 | 28 | >100 |
| | | mutant | 2 | 19 | 24 | 44 | 50 | 71 | 43.3 |
| LNA(T) + 2 | 11 | wild-type | 3 | 10 | 14 | 19 | 21 | 28 | >100 |
| | | mutant | 13 | 26 | 36 | 51 | 59 | 73 | 26.9 |
| LNA(T)22 | 7 | wild-type | 9 | 14 | 17 | 23 | 25 | 28 | >100 |
| | | mutant | 10 | 24 | 34 | 43 | 58 | 69 | 37.2 |

TABLE 3
Effect of LNA-modified oligonucleotides on wild-type versus mutant Htt protein

| | SEQ | | | | | | | | |
|---|---|---|---|---|---|---|---|---|---|
| | ID | Htt | | | | | | | IC₅₀ |
| Oligo ID | NO | protein | 3.12 nM | 6.25 nM | 12.5 nM | 25 nM | 50 nM | 100 nM | (nM) |
| LNA(C) | 9 | wild-type | 9 | 38 | 8 | 27 | 4 | 0 | >100 |
| | | mutant | 12 | 8 | 3 | 37 | 7 | 0 | >100 |
| LNA(T) + 1 | 10 | wild-type | 0 | 4 | 6 | 4 | 6 | 3 | >100 |
| | | mutant | 0 | 19 | 18 | 22 | 26 | 36 | >100 |
| LNA(G) + 1 | 10 | wild-type | 4 | 14 | 10 | 0 | 0 | 0 | >100 |
| | | mutant | 0 | 27 | 28 | 6 | 0 | 0 | >100 |
| LNA(C) + 2 | 11 | wild-type | 3 | 6 | 0 | 0 | 0 | 22 | >100 |
| | | mutant | 2 | 6 | 0 | 0 | 2 | 42 | >100 |
| LNA(T)16 | 12 | wild-type | 0 | 10 | 14 | 3 | 1 | 11 | >100 |
| | | mutant | 0 | 15 | 15 | 3 | 24 | 26 | >100 |
| LNA(T)13 | 13 | wild-type | 4 | 1 | 2 | 0 | 5 | 0 | >100 |
| | | mutant | 0 | 2 | 12 | 2 | 17 | 0 | >100 |
| LNA-gap | 9 | wild-type | 0 | 3 | 16 | 21 | 39 | 62 | 69.2 |
| | | mutant | 0 | 7 | 26 | 41 | 63 | 83 | 32.5 |

### Example 3

### Effect of Chemically Modified Oligonucleotides, Targeting Human Huntingtin (htt) mRNA, on Huntingtin (Htt) Protein

Antisense oligonucleotides targeted to the CAG repeat sequence of mutant huntingtin nucleic acid and with various chemical modifications were tested for their effects on Htt protein levels in vitro. GM04281 fibroblast cells were cultured at a density of 60,000 cells per well in 6-well plates and transfected using Lipofectamine™ RNAiMAX reagent (Invitrogen, CA) with 100 nM antisense oligonucleotide for 24 hours. The cell samples were processed for protein analysis utilizing the procedure outlined in Example 1.

The percentage inhibition of the protein samples was calculated relative to the negative control sample and presented in Table 4. The comparative percent inhibitions of the wild-type Htt protein and the mutant Htt protein are also presented. The Tₘ value for each oligonucleotide, determined by DSC, is also shown.

The antisense oligonucleotides utilized in the assay are described in Table 4. The antisense oligonucleotides were obtained from Sigma Aldrich, ISIS Pharmaceuticals, Glen Research (Virginia, USA), or the M. J. Damha laboratory (McGill University, Montreal, Canada). The modifications in each oligonucleotide are indicated as follows: subscript ‘E’=cEt; subscript ‘l’=cLNA; subscript ‘L’=LNA; bracketed base=ENA, italicized base=MOE.

TABLE 4
Effect of chemically modified antisense oligonucleotides
on wild-type and mutant Htt protein

| | | | | % inhibition | | SEQ |
|---|---|---|---|---|---|---|
| Oligo | | | Tₘ | wild- | | ID |
| ID | Sequence | Length | (° C.) | type | mutant | NO |
| DNA22 | GCTGCTGCTGCTGCTGCTGCTG | 22 | 77 | 0 | 0 | 7 |
| (−) | GCT_{L}ATA_{L}CCA_{L}GCG_{L}TCG_{L}TCA_{L}T | 19 | 0 | 0 | 0 | 8 |
| control | | | | | | |
| cEt | GCU_{E}GCU_{E}GCU_{E}GCU_{E}GCU_{E}GCU_{E}G | 19 | 95 | 0 | 56 | 14 |
| cLNA | GCTₗGCTₗGCTₗGCTₗGCTₗGCTₗG | 19 | 0 | 0 | 73 | 9 |
| ENA | GC[T]GC[T]GC[T]GC[T]GC[T]GC[T]G | 19 | 95 | 0 | 27 | 9 |
| ENA-gap | [G][C][T][G]CTGCTGCTGCT[G][C][T][G] | 19 | 95 | 30 | 74 | 9 |
| MOE | GCTGCTGCTGCTGCTGCTG | 19 | 103 | 0 | 5 | 3 |
| MOE-cEt | GCU_{E}GCU_{E}GCU_{E}GCU_{E}GCU_{E}GCU_{E}G | 19 | 115 | 0 | 5 | 8 |

### Example 4

### In Vitro Dose-Dependent Effect of Chemically Modified Oligonucleotides on Human Htt Protein

Antisense oligonucleotides from Example 3 (see Table 4 for description of chemical modifications) were tested at various doses in patient fibroblast cells. GM04281 fibroblast cells were plated at a density of 60,000 cells per well in 6-well plates and transfected using Lipofectamine™ RNAiMAX reagent (Invitrogen, CA) reagent with increasing concentrations of antisense oligonucleotide for 24 hours, as specified in Tables 5 and 6. The cell samples were processed for protein analysis utilizing the procedure outlined in Example 1.

Results are presented in Tables 5 and 6 as percent inhibition of wild-type and mutant Htt protein, relative to untreated control cells. The data presented is an average of several independent assays performed with each antisense oligonucleotide. As illustrated in Table 4, Htt mutant protein levels were reduced in a dose-dependent manner in antisense oligonucleotide treated cells. The IC₅₀ (nM) values for each oligonucleotide for inhibition of the mutant protein and wild-type protein is also shown and indicates that each oligonucleotide preferentially targets the mutant htt mRNA compared to the wild-type. The oligonucleotides listed in Table 6 demonstrate low in vitro potency and/or little to no preferential reduction of the mutant mRNA compared to the wild-type.

TABLE 5
Dose-dependent effect of chemically-modified oligonucleotides on wild-type versus mutant Htt
protein

| | SEQ | Htt | | | | | | | IC₅₀ |
|---|---|---|---|---|---|---|---|---|---|
| Oligo ID | IDNO | protein | 3.12 nM | 6.25 nM | 12.5 nM | 25 nM | 50 nM | 100 nM | (nM) |
| cEt | 14 | wild- | 0 | 0 | 7 | 12 | 21 | 30 | >100 |
| | | type | | | | | | | |
| | | mutant | 0 | 10 | 25 | 42 | 64 | 77 | 33.3 |
| cLNA | 9 | wild- | 13 | 23 | 26 | 29 | 30 | 31 | >100 |
| | | type | | | | | | | |
| | | mutant | 19 | 42 | 47 | 60 | 69 | 73 | 15.1 |

TABLE 6
Effect of chemically modified oligonucleotides on wild-type versus mutant Htt protein

| | SEQ | | | | | | | | |
|---|---|---|---|---|---|---|---|---|---|
| | ID | Htt | | | | | | | IC₅₀ |
| Oligo ID | NO | protein | 3.12 nM | 6.25 nM | 12.5 nM | 25 nM | 50 nM | 100 nM | (nM) |
| ENA | 9 | wild-type | 0 | 9 | 20 | 13 | 15 | 17 | >100 |
| | | Mutant | 0 | 0 | 18 | 18 | 29 | 33 | >100 |
| ENA-gap | 9 | wild-type | 0 | 0 | 1 | 21 | 34 | 63 | 93.4 |
| | | mutant | 5 | 0 | 5 | 33 | 54 | 83 | 40.9 |
| MOE | | wild-type | 19 | 27 | 24 | 23 | 39 | 38 | >100 |
| | | mutant | 26 | 37 | 34 | 36 | 54 | 51 | 70 |
| MOE-cEt | | wild-type | 24 | 36 | 27 | 27 | 23 | 32 | >100 |
| | | mutant | 32 | 45 | 31 | 27 | 30 | 37 | >100 |

### Example 5

### Effect of Oligonucleotides Having Phosphorothioate Backbone, Targeting Human Huntingtin (htt) mRNA, on Huntingtin (Htt) Protein

Antisense oligonucleotides targeted to the CAG repeat sequence of mutant huntingtin nucleic acid and with uniform phosphorothioate backbone were tested for their effects on Htt protein levels in vitro. GM04281 fibroblast cells were cultured at a density of 60,000 cells per well in 6-well plates and transfected using Lipofectamine™ RNAiMAX reagent (Invitrogen, CA) with 100 nM antisense oligonucleotide for 24 hours. The cell samples were processed for protein analysis utilizing the procedure outlined in Example 1.

The percentage inhibition of the protein samples was calculated relative to the negative control sample and presented in Table 7. The comparative percent inhibitions of the wild-type Htt protein and the mutant Htt protein are also presented. The Tₘ value for each oligonucleotide, determined by DSC is also shown.

The antisense oligonucleotides utilized in the assay are described in Table 7. The antisense oligonucleotides were from ISIS Pharmaceuticals. The modifications in each oligonucleotide are indicated as follows: subscript ‘E’=cEt; subscript ‘L’=LNA; italicized base=MOE; bolded base=2′F-RNA; and mC=5-methylcytosine. LNA(T)-PS, cEt-PS, MOE-PS, and MOE-cEt-PS have phosphorothioate linkages.

TABLE 7
Effect of antisense oligonucleotides with phosphorothioate
backbone on wild-type and mutant Htt protein

| | | | | % inhibition | | SEQ |
|---|---|---|---|---|---|---|
| Oligo | | | Tₘ | wild- | | ID |
| ID | Sequence | Length | (° C.) | type | mutant | NO |
| DNA22 | GCTGCTGCTGCTGCTGCTGCTG | 22 | 77 | 0 | 0 | 7 |
| (−) | GCT_{L}ATA_{L}CCA_{L}GCG_{L}TCG_{L}TCA_{L}T | 19 | 0 | 0 | 0 | 8 |
| control | | | | | | |
| LNA(T)-PS | GCT_{L}GCT_{L}GCT_{L}GCT_{L}GCT_{L}GCT_{L}G | 19 | 90 | 19 | 60 | 9 |
| cEt-PS | GCU_{E}GCU_{E}GCU_{E}GCU_{E}GCU_{E}GCU_{E}G | 19 | 88 | 17 | 72 | 14 |
| MOE-PS | GCTGCTGCTGCTGCTGCTG | 19 | 95 | 6 | 44 | 9 |
| MOE-cEt-PS | GCU_{E}GCU_{E}GCU_{E}GCU_{E}GCU_{E}GCU_{E}G | 19 | 110 | 18 | 54 | 14 |

### Example 6

### In Vitro Dose-Dependent Effect of Oligonucleotides Having Phosphorothioate Backbone on Human Htt Protein

Antisense oligonucleotides from Example 5 (see Table 7 for description of chemical modifications) were tested at various doses in patient fibroblast cells. GM04281 fibroblast cells were plated at a density of 60,000 cells per well in 6-well plates and transfected using Lipofectamine™ RNAiMAX reagent (Invitrogen, CA) reagent with increasing concentrations of antisense oligonucleotide for 24 hours, as specified in Tables 8 and 9. The cell samples were processed for protein analysis utilizing the procedure outlined in Example 1.

Results are presented in Tables 8 and 9 as percent inhibition of wild-type and mutant Htt protein, relative to untreated control cells. The data presented is an average of several independent assays performed with each antisense oligonucleotide. As illustrated in Table 8, Htt mutant protein levels were reduced in a dose-dependent manner in antisense oligonucleotide treated cells. The IC₅₀ (nM) values for each oligonucleotide for inhibition of the mutant protein and wild-type protein is also shown and indicates that each oligonucleotide preferentially targets the mutant htt mRNA compared to the wild-type. The oligonucleotides listed in Table 9 do not show preferential targeting of the mutant mRNA compared to the wild-type.

TABLE 8
Dose-dependent effect of oligonucleotides with phosphorothioate backbone on wild-type versus
mutant Htt protein

| | SEQ ID | Htt | | | | | | | IC₅₀ |
|---|---|---|---|---|---|---|---|---|---|
| Oligo ID | NO | protein | 3.12 nM | 6.25 nM | 12.5 nM | 25 nM | 50 nM | 100 nM | (nM) |
| LNA(T)-PS | 9 | wild-type | 1 | 9 | 16 | 19 | 31 | 57 | 87.1 |
| | | mutant | 19 | 37 | 53 | 61 | 69 | 82 | 13.9 |
| cEt-PS | 14 | wild-type | 0 | 6 | 12 | 21 | 34 | 71 | 63.9 |
| | | mutant | 9 | 28 | 45 | 64 | 73 | 90 | 15.9 |

TABLE 9
Effect of oligonucleotides with phosphorothioate backbone on wild-type versus mutant Htt
protein

| | SEQ ID | Htt | | | | | | | IC₅₀ |
|---|---|---|---|---|---|---|---|---|---|
| Oligo ID | NO | protein | 3.12 nM | 6.25 nM | 12.5 nM | 25 nM | 50 nM | 100 nM | (nM) |
| MOE-PS | 9 | wild-type | 16 | 25 | 32 | 40 | 58 | 72 | 33.8 |
| | | mutant | 15 | 18 | 24 | 48 | 68 | 77 | 28.3 |
| MOE-cEt- | 14 | wild-type | 19 | 26 | 26 | 36 | 44 | 54 | 80.2 |
| PS | | mutant | 23 | 33 | 41 | 51 | 60 | 68 | 24.2 |

### Example 7

### Effect of Chemically Modified Oligonucleotides Targeting CAG Repeats on Human Huntingtin (htt) mRNA Levels

Antisense oligonucleotides from Example 1, Example 3, and Example 5 (see Table 1, Table 4, and 7, respectively, for description of chemical modifications) were tested in GM04281 cells. Antisense oligonucleotides targeted to the CAG repeat sequence of mutant huntingtin nucleic acid and with various chemical modifications were tested for their effects on htt mRNA levels in vitro. GM04281 cells were cultured at a density of 60,000 cells per well in 6-well plates and transfected using Lipofectamine™ RNAiMAX reagent (Invitrogen, CA) with 50 nM antisense oligonucleotide for 24 hours. The wells were then aspirated and fresh culture medium was added to each well. After a post-transfection period of 3 days, the cells were harvested with trypsin solution (0.05% Trypsin-EDTA, Invitrogen) and lysed.

Total RNA from treated and untreated fibroblast cells was extracted using TRIzol reagent (Invitrogen). Samples were then treated with DNase I (Worthington Biochemical Corp.) at 25° C. for 10 min. Reverse transcription reactions were carried out using a High Capacity cDNA Reverse Transcription Kit (Applied Biosystems) according to the manufacturer's protocol. Quantitative PCR was performed on a BioRad CFX96 Real Time System using iTaq SYBR Green Supermix with ROX (Bio-rad). Data was normalized relative to GAPDH mRNA levels. Primer sequences specific for htt are as follows: forward primer, 5′-CGACAGCGAGTCAGTGAATG-3′ (designated herein as SEQ ID NO: 15) and reverse primer, 5′-ACCACTCTGGCTTCACAAGG-3′ (designated herein as SEQ ID NO: 16). Primers specific for GAPDH were obtained from Applied Biosystems.

The results are presented in Table 10 and indicate the percent inhibition of htt mRNA compared to untreated cells. The results indicate that mRNA levels were unaffected by treatment with the antisense oligonucleotides.

TABLE 10
Effect of antisense oligonucleotides targeting
CAG repeats on htt mRNA levels

| Oligo ID | SEQ ID NO | % inhibition |
|---|---|---|
| (−) control | 8 | 7 |
| LNA-gap | 9 | 35 |
| LNA(T) | 9 | 8 |
| LNA(G) | 9 | 0 |
| LNA(T) + 2 | 11 | 15 |
| LNA(T)22 | 7 | 7 |
| cEt | 14 | 16 |
| LNA(T)-PS | 9 | 0 |
| cEt-PS | 14 | 0 |

### Example 8

### Time-Dependent Effect of an Lna-Modified Antisense Oligonucleotide, Targeting Mutant htt mRNA, on Human Huntingtin (Htt) Protein Levels

The ISIS antisense oligonucleotide with LNA modifications at the thymine bases and which demonstrated significant selective inhibition of mutant huntingtin protein compared to wild-type protein (see Tables 1 and 2 for description of chemical modifications) was further studied. The time-dependent effect of this oligonucleotide was tested. GM04281 fibroblast cells were cultured at a density of 60,000 cells per well in 6-well plates and transfected using Lipofectamine™ RNAiMAX reagent (Invitrogen, CA) with 100 nM antisense oligonucleotide for 24 hours. The cell samples were processed for protein analysis at 2 days, 3 days, 4 days, 5 days, and 6 days post-transfection, utilizing the procedure outlined in Example 1.

The results are presented in Table 11 and indicate the preferential time-dependent decrease in mutant huntingtin protein levels. The effect was observed to be optimal at day 3 post-transfection.

TABLE 11
Time-dependent effect of LNA-modified ISIS antisense
oligonucleotide on mutant Htt protein levels

| Time (days) | wild-type | mutant |
|---|---|---|
| 2 | 19 | 52 |
| 3 | 30 | 61 |
| 4 | 14 | 49 |
| 5 | 9 | 35 |
| 6 | 18 | 32 |

### Example 9

### Oligonucleotide Selectivity of Mutant Huntingtin mRNA Containing 41 or 44 Repeat Lengths

Studies in Examples 1-8, describing oligonucleotide selectivity for the mutant allele versus the wild-type allele of the htt gene, were performed in the GM04281 fibroblast cell line, which contains 69 CAG repeats in the mutant htt allele. In order to determine whether the antisense oligonucleotides would selectively target mutant htt mRNA with shorter CAG repeats, two HD patient-derived fibroblast cell lines, GM04717 and GM04719, (Corriell Institute for Medical Research, NJ, USA), were utilized. The GM04717 fibroblast cell line contains 41 repeats on the mutant allele and 20 repeats on the wild-type allele. The GM04719 fibroblast cell line contains 44 repeats on the mutant allele and 15 repeats on the wild-type allele.

Cells were maintained at 37° C. and 5% CO₂ in MEM (Sigma) supplemented with 10% FBS (Sigma) and 0.5% MEM nonessential amino acids (Sigma). Cells were plated in 6-well dishes at 60,000 cells/well in supplemented MEM two days before transfection. Stock solutions of modified antisense oligonucleotides were heated at 65° C. for 5 minutes prior to use to dissolve any aggregation. Modified ASOs were transfected into cells at varying doses, described below in Tables 12 and 13, using Lipofectamine™ RNAiMAX (Invitrogen, USA), according to the manufacturer's instructions. Media was exchanged one day after transfection with fresh supplemented media. Cells were washed with PBS and harvested four days after transfection for protein analysis.

Cells were harvested with trypsin-EDTA solution (Invitrogen) and lysed. The protein concentration in each sample was quantified with micro-bicinchoninic acid (micro-BCA) assay (Thermo Scientific). SDS-PAGE (separating gel: 5% acrylamide-bisacrylamide [50:1], 450 mM Tris-acetate pH 8.8; stacking gel: 4% acrylamide-bisacrylamide [50:1], 150 mM Tris-aceate pH 6.8) was used to separate wild-type and mutant HTT proteins. Gels were run at 30 mA per gel for 6-7 hours in Novex Tris-acetate SDS Running Buffer (Invitrogen). The electrophoresis apparatus was placed in a 15° C. water bath to prevent overheating. In parallel with analysis for HTT expression, samples were analyzed for β-actin expression by SDS-PAGE (7.5% acrylamide pre-cast gels; Bio-Rad) to ensure even loading of protein in all lanes. These gels were run at 80 V for 15 minutes followed by 100V for 1 hour in 1×TGS buffer (Bio-Rad).

After electrophoresis, proteins were transferred to membrane (Hybond-C Extra; GE Healthcare Bio-Sciences). Primary antibodies specific for HTT (MAB2166, Chemicon) and β-actin (Sigma) protein were obtained and used at 1:10,000 dilutions. HRP conjugate anti-mouse secondary antibody (1:10,000, Jackson Immuno Research Laboratories) was used for visualizing proteins using SuperSignal West Pico Chemiluminescent Substrate (Thermo Scientific). Protein bands were quantified from autoradiographs using ImageJ Software. Percentage of inhibition was calculated as a relative value to control samples.

Each data plot from dose response experiments for inhibition of HTT was fit to the following model equation: y=100 (1−x^{m}/(n^{m}+x^{m})) using Prism 4.0(GraphPad), where y is percent expression of HTT protein and x is concentration of ASO. Both m and n are fitting parameters, where n is taken as the IC₅₀ value. The IC₅₀ values were calculated from individual dose responses fit to the above equation and then reported as the mean and standard error of the mean of three or more biological replicates.

The antisense oligonucleotides tested were LNA (T) (described in Example 1) and cEt (described in Example 3). The results are presented in Tables 12 and 13. Results demonstrate that both the mutant and wild-type alleles are reduced in a dose-dependent manner. However, the mutant allele is reduced more significantly than the wild-type allele.

The IC₅₀ for each antisense oligonucleotide is presented in Table 14. The mutant allele is reduced three- to seven-fold more than the wild-type allele, demonstrating that the antisense oligonucleotide selectively reduces the mutant allele. This data indicates that allele-specific antisense oligonucleotides can discriminate between the wild-type allele and mutant allele of htt, even when the numbers of CAG repeats are 41 and 44 in number.

TABLE 12
Dose dependent inhibition and allele-selectivity of antisense oligonucleotides in GM04717
fibroblasts

| | SEQ ID | | | | | | | | |
|---|---|---|---|---|---|---|---|---|---|
| | NO | Allele | 1.00 nM | 3.125 nM | 6.25 nM | 12.5 nM | 25.00 nM | 50.00 nM | 100.00 nM |
| LNA(T) | 9 | WT | 0 | 10 | 22 | 21 | 33 | 32 | 53 |
| | | Mutant | 0 | 16 | 30 | 31 | 50 | 58 | 78 |
| cEt | 14 | WT | 0 | 8 | 18 | 19 | 23 | 26 | 31 |
| | | Mutant | 0 | 11 | 28 | 33 | 49 | 57 | 64 |

TABLE 13
Dose dependent inhibition and allele-selectivity of antisense oligonucleotides in GM04719
fibroblasts

| | SEQ ID | | | | | | | | |
|---|---|---|---|---|---|---|---|---|---|
| | NO | Allele | 1.00 nM | 3.125 nM | 6.25 nM | 12.5 nM | 25.00 nM | 50.00 nM | 100.00 nM |
| LNA(T) | 9 | WT | 0 | 6 | 11 | 22 | 34 | 28 | 32 |
| | | Mutant | 0 | 15 | 27 | 40 | 54 | 51 | 55 |
| cEt | 14 | WT | 0 | 14 | 32 | 33 | 33 | 34 | 48 |
| | | Mutant | 0 | 20 | 39 | 51 | 58 | 64 | 80 |

TABLE 14
IC₅₀ and allele-selectivity of antisense oligonucleotides in GM04717
and GM04719 fibroblasts

| | Mutant/ | | | | | |
|---|---|---|---|---|---|---|
| | WT | Oligo- | SEQ ID | IC₅₀ (nM) | | fold |
| Cell line | repeat# | nucleotide | NO | WT | Mutant | selectivity |
| GM04717 | 41/20 | LNA(T) | 9 | >100 | 27 | 4 |
| | | cEt | 14 | >100 | 34 | 3 |
| GM04719 | 44/15 | LNA(T) | 9 | >100 | 40 | 3 |
| | | cEt | 14 | >100 | 15 | 7 |

### Example 10

### Role of Different Transfection Reagents in the Efficacy of Antisense Oligonucleotides Targeting the CAG Repeat Sequence of htt mRNA

To test whether the chemistries of the different antisense oligonucleotides affect the transfection efficiency of the oligonucleotides and, hence distort their efficacy to inhibit mutant htt mRNA, a side-by-side comparison of inhibition by the antisense oligonucleotides transfected with five different transfection reagents was performed. Transfection reagents Lipofectamine™ RNAiMAX (Invitrogen, CA, USA), Oligofectamine™ (Invitrogen, CA, USA), TriFECTin (Integrated DNA Technologies, CA, USA), TransIT®-Oligo (Mirus Bio LLC, WI, USA), and PepMute™ (Signagen Laboratories, MD, USA) were utilized in this study.

The antisense oligonucleotides tested were LNA (T) (described in Example 1) and MOE (described in Example 4). A negative control LNA oligonucleotide and a positive control siRNA (siHdh1 siRNA) were also included in the assay. The antisense oligonucleotides were transfected into GM04281 cell and protein analysis of htt was done in a procedure similar to that described in Example 1. The results are presented in Table 15, below and are expressed as percent inhibition compared to the negative control.

As presented in Table 15 the LNA(T) oligonucleotide demonstrated potency and allele-specificity, regardless of the transfection reagent used. The performance of the all lipid-based transfection reagents (Lipofectamine™ RNAiMAX, Oligofectamine™, TriFECTin, and TransIT®-Oligo) were therefore similar. In previous experiments, LNA(T) showed allele-selective inhibition while the MOE oligo demonstrated less inhibition and little or no selectivity. Using other transfection reagents, the MOE ASO showed allele-selectie inhibition.

In case of the non-lipid peptide-based transfection reagent, PepMute™, it was observed that the MOE oligonucleotide, transfected into cells with this transfection reagent, demonstrated both potency and allele-specificity. Hence, the choice of transfection reagent may affect comparisons between oligonucleotide chemistries and may be the reason for an antisense oligonucleotide underperforming in a particular cellular assay.

TABLE 15
Potency (% inhibition of htt mRNA) and allele-selectivity of antisense oligonucleotides
with different transfection reagents

| | | | | TransIT- | | |
|---|---|---|---|---|---|---|
| | Allele | RNAiMax | TriFECTin | Oligo | Oligofectamine | PepMute |
| Negative | WT | 0 | 0 | 0 | 0 | 0 |
| control LNA | mutant | 0 | 0 | 0 | 0 | 0 |
| Positive control | WT | 76 | 62 | 80 | 39 | 97 |
| | mutant | 80 | 75 | 90 | 49 | 99 |
| LNA (T) | WT | 28 | 20 | 28 | 16 | 30 |
| | mutant | 54 | 44 | 58 | 32 | 76 |
| MOE | WT | 5 | 0 | 21 | 4 | 29 |
| | mutant | 17 | 16 | 35 | 17 | 77 |

### Example 11

### Effect of Antisense Oligonucleotides Targeting CAG Repeats of Mutant Htt in R6/2 Mice Via Single Intrastriatal Bolus Administration

R6/2 mice were administered ISIS oligonucleotides as a single bolus to the right striatum for the purpose of testing the selectivity of the antisense oligonucleotides against mutant huntingtin protein expression in that tissue. The antisense oligonucleotides used in this study are presented in Table 16 and 17. In Table 16, the chemistry motifs are as shown in subscripts as ‘k’=cEt and ‘d’=2′-deoxyribose. All the cytosine residues are 5-methylcytosines. Each internucleoside linkage is a phosphorothioate linkage. In Table 17, the chemistry motifs are shown in subscripts as ‘E’=cEt. All the cytosine residues are 5-methylcytosines. Each internucleoside linkage is a phosphorothioate linkage.

TABLE 16
Antisense oligonucleotides targeted to
mutant human htt mRNA

| | | SEQ |
|---|---|---|
| ISIS | | ID |
| No | Sequence | NO |
| 473813 | TₖG_{d}C_{d}TₖG_{d}C_{d}TₖG_{d}C_{d}TₖG_{d}C_{d}TₖG_{d}C_{d}TₖG_{d}C_{d}Tₖ | 11 |
| 473814 | TₖG_{d}C_{d}TₖG_{d}C_{d}TₖG_{d}C_{d}TₖG_{d}C_{d}TₖG_{d}C_{d}Tₖ | 17 |

TABLE 17
Antisense oligonucleotides targeted
to mutant human htt mRNA

| | | SEQ |
|---|---|---|
| ISIS | | ID |
| No | Sequence | NO |
| 473813 | T_{E}GCT_{E}GCT_{E}GCT_{E}GCT_{E}GCT_{E}GCT_{E} | 11 |
| 473814 | T_{E}GCT_{E}GCT_{E}GCT_{E}GCT_{E}GCT_{E} | 17 |

### Treatment and Surgery

A group of four 8-week old R6/2 mice were treated with ISIS 473814, delivered as a single bolus of 50 μg delivered in a volume of 2 μL to the right striatum. One 8-week old R6/2 mice was treated with ISIS 473813, delivered as a single bolus of 50 μg delivered in a volume of 2 L to the right striatum. A control group of three 8-week old R6/2 mice were treated with PBS in a similar procedure. After 4 weeks, the mice were euthanized and striatal tissue was extracted. A pair of fine curved forceps was placed straight down into the brain just anterior to the hippocampus to make a transverse incision in the cortex and underlying tissues by blunt dissection. The tips of another pair of fine curved forceps were placed straight down along the midsaggital sinus midway between the hippocampus and the olfactory bulb to make a longitudinal incision, cutting the corpus callosum by blunt dissection. The first pair of forceps was then used to reflect back the resultant corner of cortex exposing the striatum and internal capsule, and then to dissect the internal capsule away from the striatum. The second set of forceps was placed such that the curved ends were on either side of the striatum and pressed down to isolate the tissue. The first set of forceps was used to pinch off the posterior end of the striatum and to remove the striatum from the brain.

### Protein Analysis

Tissues from the right striatum immediately above the injection site were processed for protein analysis in a procedure similar to that described in Example 1. The peptide band intensities were quantified using Adobe Photoshop software. The results are presented in Table 18 as percent inhibition of soluble human htt protein compared to a non-specific band control. The results indicate that both the ISIS oligonucleotides inhibited the accumulated levels of soluble human mutant huntingtin protein.

TABLE 18
Effect of antisense inhibition on mutant
human htt protein levels in R6/2 mice

| ISIS No | % inhibition |
|---|---|
| 473813 | 45 |
| 473814 | 42 |

### Example 12

### Effect of Antisense Oligonucleotides Targeting CAG Repeats of Mutant Htt in R6/2 Mice Via Intracerebrovascular (ICV) Infusion

R6/2 mice were administered ISIS oligonucleotides via ICV infusion to the right striatum for the purpose of testing the selectivity of ISIS 473814 against mutant huntingtin protein expression in that tissue.

### Treatment and Surgery

A group of five 7-week old R6/2 mice was administered ISIS 473814 at 75 μg/day delivered ICV with Alzet 2002 pumps at the rate of 0.5 L/hr for 2 weeks. A control group of 4-week old R6/2 mice was similarly treated with PBS. Alzet osmotic pumps (Model 2002) were assembled according to manufacturer's instructions. Pumps were filled with a solution containing the antisense oligonucleotide and incubated overnight at 37° C., 24 hours prior to implantation. Animals were anesthetized with 3% isofluorane and placed in a stereotactic frame. After sterilizing the surgical site, a midline incision was made over the skull, and a subcutaneous pocket was created over the back, in which a pre-filled osmotic pump was implanted. A small burr hole was made through the skull above the right lateral ventricle. A cannula, connected to the osmotic pump via a plastic catheter, was then placed in the ventricle and glued in place using Loctite adhesive. The incision was closed with sutures. Antisense oligonucleotide or PBS was infused for 14 days, after which animals were euthanized according to a humane protocol approved by the Institutional Animal Care and Use Committee. Tissues from the right striatum immediately adjacent to the injection site were extracted for further analysis.

### Protein Analysis

Tissues from the right striatum immediately above the injection site were processed for protein analysis in a procedure similar to that described in Example 1. The peptide band intensities were quantified using Adobe Photoshop software. The results were expressed percent inhibition of soluble human htt protein compared to a GAPDH band. It was observed that ISIS 473814 inhibited the accumulated levels of soluble human mutant huntingtin protein by 30% compared to the PBS control.

### Example 13

### Design of Antisense Oligonucleotides Targeting CUG Repeats

Antisense oligonucleotides were designed targeting mRNA transcripts that contain multiple CUG repeats. The chemistry of these oligonucleotides as well as their sequence is shown in Tables 19 and 20. In Table 19, the symbols designated to the sugar type are shown after the base in subscript and are as follows: d=2′-deoxyribose; k=(S)-cEt; and 1=LNA (Locked Nucleic Acids). The heterocycle names are defined with standard symbols for adenine, cytosine, thymine and guanine, and ‘mC’ for 5-methylcytosine. Linkers are shown after the sugar type in subscript and designated with the following symbol: s=thioate ester, also, phosphorothioate. In Table 20, the symbols designated to the sugar type are shown after the base in subscript and are as follows: E=(S)-cEt and L=LNA (Locked Nucleic Acids). The heterocycle names are defined with standard symbols for adenine, cytosine, thymine and guanine, and ‘mC’ for 5-methylcytosine. Linkers are shown after the sugar type in subscript and designated with the following symbol: s=thioate ester, also, phosphorothioate.

TABLE 19
Design of antisense oligonucleotides targeting CUG repeats

| | | | | SEQ |
|---|---|---|---|---|
| ISIS | | | | ID |
| No | Sequence | Chemistry | Backbone | NO |
| 431896 | G_{ds}C_{ds}AₗₛG_{ds}C_{ds}AₗₛG_{ds}C_{ds}Aₗₛ | Deoxy and | Phosphorothioate | 18 |
| | G_{ds}C_{ds}AₗₛG_{ds}C_{ds}AₗₛG_{ds}C_{ds}AₗₛG_{d} | LNA units | | |
| 473810 | AₖₛG_{ds}mC_{ds}AₖₛG_{ds}mC_{ds}AₖₛG_{ds}mC_{ds} | Deoxy and | Phosphorothioate | 19 |
| | AₖₛG_{ds}mC_{ds}AₖₛG_{ds}mC_{ds}AₖₛG_{ds}mC_{ds}Aₖ | (S)-cEt | | |
| | | units | | |
| 473811 | AₖₛG_{ds}mC_{ds}AₖₛG_{ds}mC_{ds}AₖₛG_{ds} | Deoxy and | Phosphorothioate | 20 |
| | mC_{ds}AₖₛG_{ds}mC_{ds}AₖₛG_{ds}mC_{ds}Aₖ | (S)-cEt | | |
| | | units | | |

TABLE 20
Design of antisense oligonucleotides targeting
CUG repeats

| | | | | SEQ |
|---|---|---|---|---|
| ISIS | | | | ID |
| No | Sequence | Chemistry | Backbone | NO |
| 431896 | GₛCₛA_{Ls}GₛCₛA_{Ls}GₛCₛA_{Ls} | Deoxy and | Phosphorothioate | 18 |
| | GₛCₛA_{Ls}GₛCₛA_{Ls}GₛCₛA_{Ls}G | LNA units | | |
| 473810 | A_{Es}GₛmCₛA_{Es}GₛmCₛA_{Es}GₛmCₛ | Deoxy and | Phosphorothioate | 19 |
| | A_{Es}GₛmCₛA_{Es}GₛmCₛA_{Es}GₛmCₛA_{E} | (S)-cEt | | |
| | | units | | |
| 473811 | A_{Es}GₛmCₛA_{Es}GₛmCₛA_{Es}Gₛ | Deoxy and | Phosphorothioate | 20 |
| | mCₛA_{Es}GₛmCₛA_{Es}GₛmCₛA_{E} | (S)-cEt | | |
| | | units | | |

### Example 14

### Effect of BNA-Modified Antisense Oligonucleotides, Targeting Human Ataxin-3 (atx3) mRNA, on Ataxin-3 (ATX3) Protein

Antisense oligonucleotides targeted to the CAG repeat sequence of ataxin-3 mRNA and with BNA modifications were tested for their effect on ATX3 protein levels in vitro. The GM06151 fibroblast cell line (Coriell Institute for Medical Research, NJ, USA) containing 74 CAG repeats in the mutant atx3 allele and 24 CAG repeats in the wild-type allele, was utilized in this assay. The cells were maintained at 37° C. and 5% CO₂ in MEM Eagle media (Sigma Corp.), supplemented with 10% heat-inactivated fetal bovine serum (Sigma Corp) and 0.5% MEM non-essential amino acids (Sigma Corp.). Cells were cultured at a density of 70,000 cells per well in 6-well plates two days prior to transfection. The BNA-modified antisense oligonucleotides were heated at 65° C. for 5 min, then diluted to the appropriate concentration using PepMute transfection reagent (SignaGen, Ijamsville, Md.), and then transfected at various doses to the cells, according to the manufacturer's instructions. After 24 hrs, the media were removed and replaced with fresh supplemented MEM media.

After a post-transfection period of 4 days, the cells were harvested with trypsin-EDTA solution (Invitrogen, Carlsbad) and lysed. The protein concentration in each sample was quantified with the BCA assay (Thermo Scientific, Waltham, Mass.). An SDS-PAGE gel (7.5% acrylamide pre-cast gels, Bio-Rad) was used to separate wild-type and mutant ATX3 proteins. The electrophoresis apparatus was placed in an ice-water bath to prevent overheating. In parallel with analysis for ATX3 expression, portions of each protein lysate sample were also analyzed for β-actin expression by SDS-PAGE to ensure that there had been equal protein loading of each sample.

After electrophoresis, proteins in the gel were transferred to a nitrocellulose membrane and probed with specific antibodies. Primary antibodies specific for ATX3 (MAB5360, Chemicon) and β-actin (Sigma) protein were used at 1:10,000 dilutions. HRP-conjugated anti-mouse secondary antibody (1:10,000, Jackson ImmunoResearch Laboratories) was used for visualizing proteins using SuperSignal West Pico Chemiluminescent Substrate (Thermo Scientific). Protein bands were quantified using ImageJ software (Rasband, W. S., ImageJ, U.S. National Institutes of Health, Bethesda, USA, http://rsb.info.nih.gov/ij/, 1997-2007). The dose-dependent inhibition of ATX3 protein levels is presented in Table 21 and table 22. The percentage inhibition was calculated relative to the negative control sample. The IC₅₀ of the wild-type ATX3 protein and the mutant ATX3 protein and the selectivity of the antisense oligonucleotides are presented in Table 21 and Table 22.

The antisense oligonucleotides utilized in the assay are described in Table 21 and Table 22. The antisense oligonucleotides were obtained from either Glen Research Corporation (Sterling, Va.) or ISIS Pharmaceuticals. In Table 21, the carba-LNA-modified bases are denoted by subscript ‘l’ and cET-modified bases are denoted by subscript ‘E’. In Table 22, the cET-modified bases are denoted by subscript ‘k’.

TABLE 21
Effect of BNA-modified antisense oligonucleotides
on wild-type and mutant ATX3 proteins

| | | | wild- | | |
|---|---|---|---|---|---|
| | | mutant | type | | |
| | | allele | allele | | SEQ |
| Oligo | | IC₅₀ | IC₅₀ | Selectivity | ID |
| ID | Sequence | (nM) | (nM) | (fold) | NO |
| Carba- | GCTₗGCTₗGCTₗGCTₗGCTₗGCTₗG | 25 | 55 | 2.2 | 9 |
| LNA | | | | | |
| cEt | GCU_{E}GCU_{E}GCU_{E}GCU_{E}GCU_{E}GCU_{E}G | 9 | 25 | 2.6 | 14 |

TABLE 22
Effect of BNA-modified antisense oligonucleotides
on wild-type and mutant ATX3 proteins

| | | | wild- | | |
|---|---|---|---|---|---|
| | | mutant | type | | |
| | | allele | allele | | SEQ |
| Oligo | | IC₅₀ | IC₅₀ | Selectivity | ID |
| ID | Sequence | (nM) | (nM) | (fold) | NO |
| cEt | GCUₖGCUₖGCUₖGCUₖGCUₖGCUₖG | 9 | 25 | 2.6 | 14 |

### Example 15

### Antisense Inhibition of Human Dystrophia Myotonica-Protein Kinase (DMPK) mRNA by ISIS 473810 and ISIS 473811

ISIS 473810 and ISIS 473811 (see Tables 19 and 20 for description of chemical mofications) were tested for their effects on DMPK mRNA levels in vitro. Fibroblast cell lines established from DM patients, all containing a Bpm I restriction site polymorphism located within exon 10 of the mutant allele of the DMPK gene (Hamshere et al., Proc. Natl. Acad. Sci. U.S.A., 94: 7394-7399, 1997), were utilized in this assay. Cells were cultured in normal growth media with 10% fetal bovine serum (FBS) and were transfected for 5 days without transfection reagent with 500 nM antisense oligonucleotide. The wells were then aspirated and fresh culture medium was added to each well.

One day post-transfection, the cells were harvested and RNA isolated using Trizol® reagent (Invitrogen, Carlsbad, Calif.). Reverse transcription and PCR amplification reactions were conducted for DMPK mRNA, after which the cDNA was digested with Bpm I restriction enzyme. The digested samples were then run on a Tris-borate-EDTA (TBE) gel and stained with SYBR green staining dye for 30 min for DNA band visualization. The presence of the Bpm I restriction polymorphism at only the mutant allele enabled the separation of the cDNA samples into two distinct bands of 152 base-pairs (wild-type allele) and 136 base-pairs (mutant allele). The separated DNA bands were quantified using arbitrary units and the density of the same is presented in Table 23.

TABLE 23
Percent inhibition of DMPK wild-type allele and
mutant allele RNA by antisense oligonucleotides

| | Wild-type | |
|---|---|---|
| ISIS No | allele (%) | Mutant allele (%) |
| 473810 | 12 | 13 |
| 473811 | 4 | 31 |

1.-70. (canceled)

71. A chemically-modified oligonucleotide 13 to 22 nucleobases in length and having a nucleobase sequence comprising SEQ ID NO.: 2 [TGCTGCTGCTG] and 100% complementary within a repeat region of a CAG nucleotide repeat containing RNA, wherein:
a. each T is independently a uridine or thymidine nucleoside and each comprising an independently selected high-affinity sugar modification;
b. each non-terminal G is a guanosine nucleoside comprising a 2′-deoxyribose sugar;
c. each non-terminal C is a cytidine nucleoside comprises a 2′-deoxyribose sugar; and
wherein one or both of the 5′ or 3′ terminal nucleosides of the chemically-modified oligonucleotide independently comprises one or more nuclease-resistant modifications.

72. The chemically-modified oligonucleotide of claim 71, wherein the nuclease-resistant modification is a modified sugar moiety or a modified internucleoside linkage.

73. The chemically-modified oligonucleotide of claim 72, wherein the modified sugar moiety is a bicyclic sugar moiety.

74. The chemically-modified oligonucleotide of claim 73, wherein the bicyclic sugar moiety is a 4′ to 2′ bicyclic sugar moiety.

75. The chemically-modified oligonucleotide of claim 74, wherein the bicyclic sugar moiety is a 4′-CH₂—O-2′ or 4′-CH(CH₃)—O-2′ bicyclic sugar moiety.

76. The chemically-modified oligonucleotide of claim 71, wherein each high-affinity sugar modification is a 2′-modified sugar moiety or a bicyclic sugar moiety.

77. The chemically-modified oligonucleotide of claim 71, wherein each T is independently a thymidine or uridine nucleoside comprising a 4′ to 2′ bicyclic sugar moiety.

78. The chemically-modified oligonucleotide of claim 77, wherein each 4′ to 2′ bridge independently comprises from 2 to 4 linked groups independently selected from —[C(Rₐ)(R_{b})]_{y}—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(R₁)—;
wherein:
x is 0, 1, or 2;
y is 1, 2, 3, or 4;
each Rₐ and R_{b} is, independently, H, a protecting group, hydroxyl, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₉ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₅-C₂₀ aryl, substituted C₅-C₉ aryl, acyl (C(═O)—H), substituted acyl, a heterocycle radical, a substituted heterocycle radical, C₁-C₆ aminoalkyl, substituted C₁-C₆ aminoalkyl or a protecting group.

79. The chemically modified oligonucleotide of claim 78, wherein each 4′ to 2′ bridge is independently —[C(R_{c})(R_{d})]ₙ—, —[C(R_{c})(R_{d})]ₙ—O—, —C(R_{c}R_{d})—N(Rₑ)—O— or —C(RₑR_{d})—O—N(Rₑ)—, wherein:
each R_{c} and R_{d} is independently hydrogen, halogen, substituted or unsubstituted C₁-C₆ alkyl; and
each Rₑ is independently hydrogen or substituted or unsubstituted C₁-C₆ alkyl.

80. The chemically modified oligonucleotide of claim 79, wherein for each 4′ to 2′ bridge is independently a 4′-(CH₂)_{2-2′,4}′-(CH₂)_{3-2′,4}′-CH₂—O-2′,4′-CH(CH₃)—O-2′,4′-(CH₂)₂-0-2′,4′-CH₂—O—N(Rₑ)-2′ and 4′-CH₂—N(Rₑ)—O-2′-bridge.

81. The chemically-modified oligonucleotide of claim 71, wherein each T is a thymidine nucleoside comprising a 4′-CH(CH₃)—O-2′ bicyclic sugar moiety.

82. The chemically-modified oligonucleotide of claim 71, wherein the CAG nucleotide repeat containing RNA comprises 20 or more, 30 or more or 40 or more repeats.

83. The chemically-modified oligonucleotide of claim 71, wherein the nucleosides are linked by phosphate internucleoside linkages.

84. The chemically-modified oligonucleotide of claim 71, wherein at least one of the phosphate internucleoside linkages is a phosphorothioate linkage.

85. The chemically-modified oligonucleotide of claim 71, wherein each internucleoside linkage is a phosphorothioate linkage.

85. The chemically-modified oligonucleotide of claim 81, wherein each internucleoside linkage is a phosphorothioate linkage.

86. A chemically-modified oligonucleotide 13 to 22 nucleobases in length comprising a nucleobase sequence of SEQ ID NO.: 2 [TGCTGCTGCTG] which is 100% complementary within a repeat region of a CAG nucleotide repeat containing RNA, wherein:
a. each G is a guanosine nucleoside independently comprises a high affinity sugar modification;
b. each non-terminal T is independently a uridine or thymidine nucleoside comprising a 2′-deoxyribose sugar;
c. each terminal T is independently a uridine or thymidine nucleoside comprising a 2′deoxyribose sugar or a nuclease resistant modification;
d. each non-terminal C is a cytidine nucleoside comprising a 2′-deoxyribose sugar; and
each terminal C is a cytidine nucleoside comprising either a 2′deoxyribose sugar or a nuclease resistant modification.

87. A method of selectively inhibiting the function of a mutant nucleotide repeat containing RNA in a cell, comprising contacting a cell having a mutant nucleotide repeat containing RNA with a chemically-modified oligonucleotide of claim 1.

88. A method of treating a disease associated with a CAG nucleotide repeat-containing RNA, comprising contacting a cell having a mutant nucleotide repeat containing RNA with a chemically-modified oligonucleotide 13 to 22 nucleobases in length and having a nucleobase sequence comprising SEQ ID NO.: 2 [TGCTGCTGCTG] and 100% complementary within a repeat region of a CAG nucleotide repeat containing RNA, wherein:
a. each T is independently a uridine or thymidine nucleoside and each comprising an independently selected high-affinity sugar modification;
b. each non-terminal G is a guanosine nucleoside comprising a 2′-deoxyribose sugar;
c. each non-terminal C is a cytidine nucleoside comprises a 2′-deoxyribose sugar; and
wherein one or both of the 5′ or 3′ terminal nucleosides of the chemically-modified oligonucleotide independently comprises one or more nuclease-resistant modification.

89. The method of claim 88, wherein the disease is any of Atrophin 1, Huntington's Disease, Huntington disease-like 2 (HDL2), spinal and bulbar muscular atrophy, Kennedy disease, spinocerebellar ataxia 1, spinocerebellar ataxia 12, spinocerebellar ataxia 17, Huntington disease-like 4 (HDL4), spinocerebellar ataxia 2, spinocerebellar ataxia 3, Machado-Joseph disease, spinocerebellar ataxia 6, and spinocerebellar ataxia 7.

90. The method of claim 88, wherein the disease is any of Huntington disease-like 2 (HDL2), Myotonic Dystrophy (DM1), or spinocerebellar ataxia 8.
